SEQUENCE LISTING (10 sequences)

SEQ ID NO: 1 - protein, 10 aa, synthetic construct
GYTFTSSYIN

SEQ ID NO: 2 - protein, 17 aa, synthetic construct
TINPVSGSTSYAQKFQG

SEQ ID NO: 3 - protein, 6 aa, synthetic construct
GGWFDY

SEQ ID NO: 4 - protein, 14 aa, synthetic construct
TGTSSDVGSYNYVN

SEQ ID NO: 5 - protein, 11 aa, synthetic construct
LMIYGVSKRPS

SEQ ID NO: 6 - protein, 10 aa, synthetic construct
GTFAGGSYYG

SEQ ID NO: 7 - protein, 115 aa, synthetic construct [sequence omitted]

SEQ ID NO: 8 - protein, 113 aa, synthetic construct
QSALTQPASVSGSPGQSITISCTGTSSDVGSYNYVNWYQQHPGKAPKLMIYGVSKRPSGVSNRFSGSKSGNTASLTISGLQAEDEADYYCGTFAGGSYYGVFGGGTKLTVLGQ

SEQ ID NO: 9 - protein, 445 aa, synthetic construct
QVQLVQSGAEVKKPGASVKVSCKASGYTFTSSYINWVRQAPGQGLEWMGTINPVSGSTSYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 217 aa, synthetic construct
QSALTQPASVSGSPGQSITISCTGTSSDVGSYNYVNWYQQHPGKAPKLMIYGVSKRPSGVSNRFSGSKSGNTASLTISGLQAEDEADYYCGTFAGGSYYGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2024/0325530 A1
(43) Pub. Date: Oct. 3, 2024

(54) COMBINATION THERAPIES

(71) Applicant: Versanis Bio, Inc., Oakland, CA (US)

(72) Inventors: Lloyd Berl KLICKSTEIN, Newton, MA (US); Matthias MACHACEK, Allschwil (CH)

(21) Appl. No.: 18/587,617

(22) Filed: Feb. 26, 2024

Related U.S. Application Data
(63) Continuation of application No. PCT/US2022/075545, filed on Aug. 26, 2022.
(60) Provisional application No. 63/238,068, filed on Aug. 27, 2021.
(60) Provisional application No. 63/301,012, filed on Jan. 19, 2022.
(60) Provisional application No. 63/333,351, filed on Apr. 21, 2022.

(51) Int. Cl. A61K 39/395; A61K 38/26; A61K 39/00; A61P 3/04

(52) U.S. Cl. CPC A61K 39/3955; A61K 38/26; A61P 3/04; A61K 2039/54; A61K 2039/545

(57) ABSTRACT

The disclosure relates to the combined use of an ActRII pathway agent, e.g., an ActRII receptor antibody, and a GLP-1 agonist for the treatment of metabolic disorders, including obesity. As provided herein, the combination treatment reduces fat mass and maintains or increases lean mass.

### CROSS REFERENCE TO RELATED APPLICATIONS

This application claims priority to U.S. Provisional Patent Application Nos. 63/238,068 filed Aug. 27, 2021, 63/301,012 filed Jan. 19, 2022, and 63/333,351 filed Apr. 21, 2022, the contents of which are incorporated by reference in their entirety.

### INCORPORATION BY REFERENCE OF SEQUENCE LISTING

The contents of the electronic sequence listing (VRNS_009_03WO_SeqList_ST26.xml; Size: 10,557 bytes; date of Creation: Aug. 25, 2022) are herein incorporated by reference in their entirety.

### BACKGROUND

Treatment of metabolic disorders such as obesity remains elusive as the regulation of metabolism and fat storage involves complex biological feedback systems. To date, drugs effective in regulating metabolic dysfunction often exhibit several side effects, which limit their usefulness in patient populations, and in particular, in obese patient populations, who may require long term treatment. Thus, there is a need for additional compositions and methods to treat metabolic disorders including obesity.

### SUMMARY

The disclosure relates to the dual administration of ActRII pathway agents, e.g., ActRII receptor antagonists, e.g., ActRII receptor antibodies, and glucagon-like peptide-1 receptor (GLP-1) agonists for the treatment of metabolic disorders. The combination treatments of the disclosure comprising an ActRII pathway antibody and a GLP-1 agonist were found to reduce fat mass and maintain or increase lean mass. The combination treatments provided herein may improve metabolic dysfunction by reducing fat mass, increasing lean mass, improving glycemic control, and may also improve the tolerability and/or efficacy of one or both of these agents in subjects.

Provided herein are methods of treating a metabolic disorder in a subject, comprising administering to the subject in need thereof an ActRII receptor antibody and a glucagon-like peptide-1 receptor (GLP-1) agonist. In some embodiments, the metabolic disorder is selected from the group consisting of: obesity, diabetes, metabolic syndrome, anti-psychotic drug-associated obesity, glucocorticoid-induced obesity, hypothalamic obesity associated with craniopharyngioma, Prader-Willi syndrome, and a monogenetic disorder associated with obesity. In some embodiments, the diabetes is Type I diabetes or Type II diabetes.

In some embodiments, the monogenetic disorder associated with obesity, is one of Bardet-Biedl syndrome, or obesity resulting from mutations in one or more of the genes comprising: ADCY3, ALMS1, ARL6, BBS1, BBS2, BBS4, BBS5, BBS7, BBS9, BBS10, BBS12, BDNF, CCDC28B, CEP290, CREBBP, EP300, GNAS, IER3IP1, MC3R, MKKS, MKS1, MRAP2, NTRK2, PCSK1, PHF6, POMC, SH2B1, SIM1, TMEM67, TRIM32, TTC8 and VPS13B.

In some embodiments, the treatment is useful for an obesity related co-morbidity, wherein the condition is selected from the group of: glucose intolerance, prediabetes, insulin resistance, high triglycerides, overweight associated physical impairment, osteoporosis, renal disease, obstructive sleep apnea, sexual hormones impairment, endocrine reproductive disorders, osteoarthritis, gastrointestinal cancers, dyslipidemia, hypertension, heart failure, coronary heart disease, stroke, and/or gallstones.

In some embodiments, the subject has a body mass index (BMI) of 30 or greater. In some embodiments, the subject has a BMI of 27 or greater and has one or more obesity-related co-morbidities. In some embodiments, the subject is overweight. In some embodiments, the subject is 18 years of age or older. In some embodiments, the subject is 45 years of age or older. In some embodiments, the subject is a child 0-17 years of age, inclusive.

In some embodiments, the treatment reduces body weight in the subject. In some embodiments, the treatment reduces fat mass in the subject. In some embodiments, the treatment increases lean mass in the subject. In some embodiments, the treatment reduces fat mass and increases lean mass in the subject. In some embodiments, the treatment reduces fat mass and maintains lean mass in the subject. In some embodiments, the treatment reduces central adiposity in the subject. In some embodiments, the treatment improves glycemic control in the subject.

In some embodiments, the treatment improves the safety, efficacy, and/or tolerability of the ActRII receptor antibody and/or the glucagon-like peptide-1 receptor (GLP-1) agonist.

In some embodiments, the efficacy of the treatment is measured by at least one of the following: body weight; bioelectrical impedance analysis (BIA); dual X-ray absorptiometry (DXA); waist circumference; decreased BMI; waist to hip ratio; wait to height ratio; blood lipids profile; leptin, adiponectin, and adipsin levels; urine biomarkers; hemoglobin A1c (HgbA1c) levels; hand dynamometry demonstrating muscle strength; glucose levels; insulin levels; short physical performance battery (SPPB); Impact of Weight on Quality of Life (IWQoL-Lite for CT) assessment; Short Form (36) Health Survey (SF-36) assessment; homeostasis model assessment 2 (HOMA2); and physical activity monitoring via actigraphy.

In some embodiments, the antibody comprises the amino acid sequence of SEQ ID NOS: 1-6. In some embodiments, the antibody comprises the amino acid sequence of SEQ ID NO: 7, or a sequence with sequence identity of at least 90% thereto; and comprises the amino acid sequence of SEQ ID NO: 8, or a sequence with sequence identity of at least 90% thereto. In some embodiments, the antibody comprises the amino acid sequence of SEQ ID NO: 9, or a sequence with sequence identity of at least 90% thereto; and comprises the amino acid sequence of SEQ ID NO: 10, or a sequence with sequence identity of at least 90% thereto.

In some embodiments, the antibody is specific for ActRIIA and ActRIIB.

In some embodiments, the GLP-1 agonist is an antibody, small molecule, peptide, or aptamer. In some embodiments, the GLP-1 agonist is selected from the group consisting of exenatide, exenatide extended-release, dulaglutide, liraglutide, lixisenatide, semaglutide, tirzepatide, cotadutide, noiiglutide, oxyntomodulin (e.g., mazdutide), retatrutide, albiglutide, beinaglutide and PEG-loxenatide, pemvidutide, and danuglipron.

In some embodiments, the GLP-1 agonist is a dual GLP-1 agonist and GIP agonist. In some embodiments, the GLP-1 agonist is a dual GLP-1 agonist and GCG agonist. In some embodiments, the GLP-1 agonist is a triagonist of GIP/GLP-1/glucagon receptors.

In some embodiments, the ActRII receptor antibody is administered in a dose of about 3 mg/kg to about 50 mg/kg. In some embodiments, the ActRII receptor antibody is administered in a dose of about 10 mg/kg to about 30 mg/kg. In some embodiments, the ActRII receptor antibody is administered in a dose of about 10 mg/kg. In some embodiments, the ActRII receptor antibody is administered in a dose of about 30 mg/kg. In some embodiments, the ActRII receptor antibody is administered in a dose of about 200 mg to about 400 mg once weekly.

In some embodiments, the ActRII receptor antibody is administered in a dose of about 300 mg once weekly.

In some embodiments, the ActRII receptor antibody administration includes the administration of at least one loading dose of an ActRII receptor antibody at day 0 or week 0, prior to the administration of the ActRII receptor antibody and the GLP-1 agonist. In some embodiments, the loading dose of the ActRII receptor antibody is administered in a dose of about 10 mg/kg to about 30 mg/kg.

In some embodiments, the ActRII receptor antibody is administered at least once a day, at least once a week, at least twice a week, at least thrice a week, at least once every 2 weeks, at least once every 4 weeks, at least once every 6 weeks, or at least once every 12 weeks. In some embodiments, the ActRII receptor antibody is administered at week 0, at about week 4, and at least once every 12 weeks thereafter.

In some embodiments, the GLP-1 agonist is administered in a dose of about 0.005 mg to about 3.0 mg weekly. In some embodiments, the GLP-1 agonist dose is increased incrementally weekly from according to of the following regimens: from about 0.2 mg to about 3.0 mg, from about 0.1 to about 1.0 mg, from about 0.25 mg to about 2.4 mg, and from about 0.25 mg to about 0.5 mg. In some embodiments, the GLP-1 agonist dose is administered weekly in a dose of about 0.25 mg from about week 0 to about week 4, and in a dose of about 0.5 mg at week 5 and thereafter.

In some embodiments, the GLP-1 agonist dose is administered weekly in a dose of about 0.25 mg from about week 0 to about week 4, in a dose of about 0.5 mg from about week 5 to about week 8, in a dose of about 1.0 mg from about week 8 to about week 12, in a dose of about 1.7 mg from about week 12 to about week 15, in a dose of about 2.4 mg from about week 16 to about week 20, and a dose of about 2.4 mg from about week 20 and thereafter.

In some embodiments, the GLP-1 agonist dose is administered weekly in a dose of about 5.0 mg, about 10 mg, or about 15 mg.

In some embodiments, the ActRII receptor antibody and/or GLP-1 agonist are administered intravenously. In some embodiments, the ActRII receptor antibody and/or GLP-1 agonist are administered subcutaneously.

In some embodiments, the ActRII receptor antibody is administered prior to the GLP-1 agonist. In some embodiments, the ActRII receptor antibody is administered at least 12 weeks prior, at least 10 weeks prior, at least 8 weeks prior, at least 6 weeks prior, at least 4 weeks prior, at least 2 weeks prior, at least 1 week prior, at least 1 day prior, or at least 1 hour prior to the administration of the GLP-1 agonist.

In some embodiments, the GLP-1 agonist is administered prior to the ActRII receptor antibody. In some embodiments, the GLP-1 agonist is administered at least 2 weeks prior, at least 1 week prior, at least 5 days prior, at least 4 days prior, at least 2 days prior, at least 1 day prior, at least 6 hours prior, or at least 1 hour prior to the ActRII receptor antibody. In some embodiments, the ActRII receptor antibody and the GLP-1 agonist are co-administered.

Also provided herein is a combination comprising an ActRII receptor antibody and a GLP-1 agonist.

In some embodiments, the ActRII receptor antibody comprises the amino acid sequence of SEQ ID NOS: 1-6. In some embodiments, the ActRII receptor antibody comprises the amino acid sequence of SEQ ID NO: 7, or a sequence with sequence identity of at least 90% thereto; and comprises the amino acid sequence of SEQ ID NO: 8, or a sequence with sequence identity of at least 90% thereto. In some embodiments, the ActRII receptor antibody comprises the amino acid sequence of SEQ ID NO: 9, or a sequence with sequence identity of at least 90% thereto; and comprises the amino acid sequence of SEQ ID NO: 10, or a sequence with sequence identity of at least 90% thereto.

In some embodiments, the ActRII receptor antibody comprises a human IgG1 Fc domain with a modification selected from the group consisting of 259I, 252Y, 307Q, 308F, 428L, 434H, 434F, 434Y, 434A, 434M, and 434S, relative to a human IgG1 Fc domain according to the EU numbering scheme. In some embodiments, the ActRII receptor antibody comprises a human IgG1 Fc domain with a modification selected from M428L and/or N434S relative to a human IgG1 Fc domain according to the EU numbering scheme.

Also provided herein is a pharmaceutical composition comprising the combination of an ActRII receptor antibody and a GLP-1 agonist, and a pharmaceutically acceptable excipient.

### BRIEF DESCRIPTION OF THE DRAWINGS

FIG. 1 is a schematic of a clinical study designed to compare the effects of an exemplar ActRII receptor antibody, bimagrumab, and a glucagon-like peptide-1 receptor (GLP-1) agonist, semaglutide, on fat loss, when given in combination or singly. The schematic includes a loading dose at week=0.

FIG. 2 is an outline of the pre-clinical study of the effects of an ActRII receptor antibody and GLP1 agonists in diet-induced obese mice.

FIG. 3 is a bar graph of the effects of the GLP1 agonist semaglutide and an ActRII receptor antibody, alone and in combination, on circulating Activin A in diet-induced obese mice.

FIGS. 4A-4C are bar graphs of the effects of the GLP1 agonist semaglutide and an ActRII receptor antibody, alone and in combination, on fat mass measured by MRI, inguinal fat weight measured post mortem at the end of the study, and circulating leptin levels in diet-induced obese mice.

FIG. 5 is a bar graph of the effects of the GLP1 agonist semaglutide and an ActRII receptor antibody, alone and in combination, on lean muscle mass in diet-induced obese mice, assessed by weighing a gastrocnemius muscle post-mortem at the end of the study.

FIG. 6 is a chart of mRNA transcript level changes, as assessed by RNAseq, in response to the combination of the GLP1 agonist semaglutide and an ActRII receptor antibody, in the inguinal fat of diet-induced obese mice.

FIG. 7 is a line plot of the effects of the GLP1 agonists semaglutide, tirzepatide, and liraglutide, alone and in combination with an ActRII receptor antibody, on body weight over time, with a change in dose of tirzepatide at day 11 in both tirzepatide groups to induce better food intake, in diet-induced obese mice.

FIG. 8 is a line plot of the effects of the GLP1 agonists semaglutide, tirzepatide, and liraglutide, alone and in combination with an ActRII receptor antibody, on body fat over time measured by MRI in diet-induced obese mice.

FIG. 9 a bar graph of the effects of the GLP1 agonists semaglutide, tirzepatide, and liraglutide, alone and in combination with an ActRII receptor antibody, on the weight of peri-renal fat collected at the end of the study in diet-induced obese mice.

FIG. 10 is a line plot of the effects over time of the GLP1 agonists semaglutide, tirzepatide, and liraglutide, alone and in combination with an ActRII receptor antibody, on food intake in diet-induced obese mice.

FIG. 11 is a line plot of the effects over time of the GLP1 agonists semaglutide, tirzepatide, and liraglutide, alone and in combination with an ActRII receptor antibody, on lean mass in diet-induced obese mice.

FIG. 12A is a line plot of the effects of the GLP1 agonists semaglutide, tirzepatide, and liraglutide, alone and in combination with an ActRII receptor antibody, on distance traveled in quantitative open field tests of diet-induced obese mice.

FIG. 12B is a line plot of the effects of the GLP1 agonists semaglutide, tirzepatide, and liraglutide, alone and in combination with an ActRII receptor antibody, on speed in quantitative open field tests of diet-induced obese mice.

FIG. 13 is a line plot of the effects of the GLP1 agonist tirzepatide, alone and in combination with an ActRII receptor antibody, on fat mass in diet-induced obese mice.

FIG. 14 is a line plot of the effects of the GLP1 agonist tirzepatide, alone and in combination with an ActRII receptor antibody, on lean mass in diet-induced obese mice.

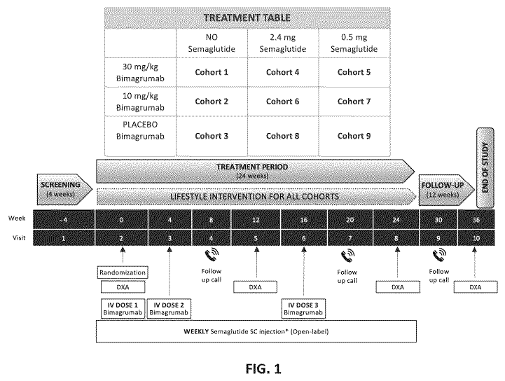

### DETAILED DESCRIPTION

The compositions and methods of the disclosure provide combinations comprising an ActRII pathway agent, e.g., an ActRII receptor antagonist, e.g., an ActRII receptor antibody, and a glucagon-like peptide-1 receptor (GLP-1) agonist for use in treating metabolic disorders, including obesity. As provided herein, the combinations significantly reduce fat mass and maintain or increase lean mass.

Both ActRII receptor antagonists and GLP-1 agonists cause significant decreases in fat mass, greater than 20% in 1 year, in human subjects. However, while ActRII receptor antagonists administered alone also increase lean mass, GLP-1 agonists administered alone provide die disadvantageous effect of decreasing lean mass.

The present disclosure demonstrates the surprising effect that combinations of an ActRII receptor antagonist and glucagon-like peptide-1 receptor (GLP-1) agonists resulted in significant and synergistic reductions in fat mass, while at the same time, maintaining or increasing lean mass.

### I. Definitions

Unless otherwise defined herein, scientific and technical terms used herein shall have the meanings that are commonly understood by those of ordinary skill in the art. Generally, nomenclature used in connection with, and techniques of, chemistry, molecular biology, cell biology, immunology, pharmacology, and protein chemistry, described herein, are those well-known and commonly used in the art.

It must be noted that, as used herein and in the appended claims, the singular forms “a,” “and,” and “the” include plural referents unless the context clearly dictates otherwise. Thus, for example, reference to “an agent” refers to one or mixtures of such candidates, and reference to “a method” includes reference to equivalent steps and methods known to those skilled in the art, and so forth.

As used herein, the term “approximately” or “about,” as applied to one or more values of interest, refers to a value that is similar in magnitude and/or within a similar range to a stated reference value. In certain embodiments, the term “approximately” or “about” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

As used herein, the teens “polypeptide,” “peptide,” and “protein” refer to polymers of amino acids of any length. The terms also encompass an amino acid polymer that has been modified; for example, to include disulfide bond formation, glycosylation, lipidation, phosphorylation, or conjugation with a labeling component.

As used herein, the terms “identity” and “identical,” when referring to a comparison of two sequences, refers to the percentage of exact matching residues in an alignment of a sequence provided herein to a reference sequence, such as an alignment generated by a BLAST algorithm or other alignment algorithms known in the art. Identity may be calculated based on an alignment of a full-length sequence provided herein and a full length reference sequence. Identity may also be calculated based on a partial alignment of a sequence provided herein and a reference sequence, if the reference sequence is longer than a sequence provided herein. Identity may also be calculated based on a partial alignment of a sequence provided herein and a reference sequence, if the reference sequence is shorter than a sequence provided herein. Thus, when aligning two sequences, according to the aforementioned, a query sequence “shares at least x % identity to” a subject sequence if in the alignment of the two sequences, at least x % (rounded down) of the residues in the subject sequence are aligned as an exact match to a corresponding residue in the query sequence, wherein the numerator is the number of exact matches and the denominator is the length of the query sequence. In some embodiments, the denominator may alternatively be the length of the query sequence minus any gaps of two or more non-matching residues. Where the subject sequence has variable positions (e.g., residues denoted X), an alignment to any residue in the query sequence is counted as a match.

The terms “treatment”, “treating” and the like are used herein to generally mean obtaining a desired pharmacologic and/or physiologic effect with a therapeutic agent. The effect may be prophylactic in terms of completely or partially preventing a disease or symptom thereof, e.g., reducing the likelihood that the disease or symptom thereof occurs in the subject, and/or may be therapeutic in terms of completely or partially reducing a symptom, or a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting or slowing the onset or development of the disease; or (c) relieving the disease, e.g., causing regression of the disease or symptoms associated with the disease. The therapeutic agent may be administered before, during or after the onset of disease. The treatment of ongoing disease, where the treatment stabilizes or reduces the undesirable clinical symptoms of the patient, may be of particular interest. In some embodiments, treatment is performed prior to complete loss of function in the affected tissues. In some embodiments, the subject therapy will be administered during the symptomatic stage of the disease, and in some embodiments, after the symptomatic stage of the disease.

The term “loading dose” refers to one or more doses of a therapeutic agent that are administered in addition to the combination therapies provided herein. As used herein a “loading dose” may refer to one or more doses of an ActRII receptor antibody which are the same concentration, a lower concentration, or a higher concentration than the doses of an ActRII receptor antibody that are a part of the combination therapy.

The terms “individual.” “subject,” and “patient” are used interchangeably herein and refer to any subject for whom treatment or therapy is desired. The subject may be a mammalian subject. Mammalian subjects include, e.g., humans, non-human primates, rodents, (e.g., rats, mice), lagomorphs (e.g., rabbits), ungulates (e.g., cows, sheep, pigs, horses, goats, and the like), etc. In some embodiments, the subject is a human. In some embodiments, the subject is a non-human primate, for example a cynomolgus monkey. In some embodiments, the subject is a companion animal (e.g., cats, dogs).

A metabolic disorder as used herein refers to a disorder affecting dysregulation of mammalian metabolism, including but not limited to: obesity, diabetes (Type I and II), metabolic syndrome, anti-psychotic drug-associated obesity, glucocorticoid-induced obesity, hypothalamic obesity associated with craniopharyngioma, and monogenetic disorder associated obesity. The monogenetic disorders associated with obesity in humans, may include but are not limited to Bardet-Biedl syndrome, and a disorder arising from a mutation in one or more of the following genes ADCY3, ALMS 1, ARL6, BBS1, BBS2. BBS4, BBS5, BBS7, BBS9, BBS10, BBS12, BDNF, CCDC28B, CEP290, CREBBP, EP300, GNAS, IER31P1, MC3R, MKKS, MKS1, MRAP2, NTRK2, PCSK1, PHF6, POMC, SH2B1, SIM1, TMEM67, TRIM32, TTC8 and VPS13B, or combinations thereof. A metabolic disorder may also be associated with a complex genetic disorder, e.g., Prader-Willi syndrome.

As used herein, Body Mass Index or “BMI” is calculated as weight in kilograms (kg) divided by height in meters squared (m²), rounded to one decimal place. As used herein, “obesity” in adult humans is defined as a BMI greater than or equal to 30 kg/m². “Obesity” in human youth is defined as a BMI greater than or equal to the age- and sec-specific 95th percentile of the 2000 CDC growth chaps. The term “overweight” is defined as a BMI of greater than or equal to 25 and less than 30.

The terms “obesity related co-morbidity,” “obesity related condition” and “obesity related disorder” may be used interchangeably and refer to a health condition depending from the obesity of the subject. In some embodiments, the obesity related co-morbidity or condition increases the mortality risk of the subject. Obesity related co-morbidities include but are not limited to: high blood pressure (hypertension), high LDL cholesterol, low HDL cholesterol, high levels of triglycerides (dyslipidemia), Type 2 diabetes, coronary heart disease, stroke, gallbladder disease, osteoarthritis, sleep apnea, breathing problems, cancer, gastroesophageal reflex disease, severe COVID-19, overall mortality, lower quality of life, mental illness such as clinical depression, anxiety, and other mental disorders, and body pain and difficulty with physical functioning.

“Lean mass” is defined as total body mass of a subject minus the fat mass and minus the bone mass of the subject. Lean mass and fat mass may be measured by, for example, bioelectrical impedance analysis (BIA), magnetic resonance imaging (MRI) or dual X-ray absorptiometry (DXA).

As used herein, “antibody” includes reference to an immunoglobulin molecule immunologically reactive with a particular antigen, and includes both polyclonal and monoclonal antibodies. The term includes humanized antibodies, chimeric antibodies e.g., murine variable region with a human constant region) and conjugated antibodies. The term “antibody” also includes antigen binding forms of antibodies, including fragments that retain antigen-binding capability (e g., Fab′, F(ab′)₂, Fab, single chain variable fragments (scFv) containing VH and VL sequences linked together in one chain, single chain antibody fragments (scAb). Fragment crystallizable regions (Fc) may also be linked to any of the aforementioned antigen-binding fragments. The term antibody also includes bivalent or bispecific molecules, diabodies, triabodies, and tetrabodies.

### II. ActRII Pathway Modulators

### ActRII Receptor Antibodies

Activin receptor II B (ActRIIB) is a receptor for myostatin, activin, and bone morphogenetic proteins (BMPs). The interaction between myostatin and this receptor regulates the inhibition of skeletal muscle differentiation via the Smad-dependent pathway. It is thought that by inhibiting or preventing myostatin from binding to ActRIIB, e.g., via an ActRII receptor antibody, the formation of skeletal muscle can be induced. Regulation of the activin receptor II A (ActRIIA) also plays a role in the regulation of muscle growth (Morvan et al. 2017).

Exemplary ActRII receptor antibodies that bind to ActRIIA and ActRIIB were shown to not only increase lean muscle mass, but also to decrease fat mass and improve glycemic control in human clinical studies (WO2010125003A1, WO2018116201A1, the contents of which are incorporated in their entirety, and Heymsfield et al. 2021; 4(1):e2033457, JAMA). Exemplary ActRII receptor antibodies that may be used in some embodiments include the human recombinant antibodies, isolated and structurally characterized, as described in WO2010125003A1.

In some embodiments, exemplary antibodies of the disclosure include the sequences of Bimagrumab (BYM338). The below table provides the relevant CDR, VH, VL, HC, and LC amino acid sequences for Bimagrumab.

Bimagrumab Sequences
Bimagrumab Heavy Chain Complementarity
Determining Regions (CDRs)
CDRH1-
(SEQ ID NO: 1)
GYTFTSSYIN
CDRH2-
(SEQ ID NO: 2)
TINPVSGSTSYAQKFQ
CDRH3-
(SEQ ID NO: 3)
GGWFDY
Bimagrumab Light Chain Complementarity
Determining Regions (CDRs)
CDRL1-
(SEQ ID NO: 4)
TGTSSDVGSYNYVN
CDRL2-
(SEQ ID NO: 5)
MIYGVSKRPS
CDRL3-
(SEQ ID NO: 6)
GTFAGGSYYG
Bimagrumab Variable Heavy Chain (VH)
(SEQ ID NO: 7)
QVQLVQSGAEVKKPGASVKVSCKASGYTFTSSYINWVRQAPGQGL
EWMGTINPVSGSTSYAQKFQGRVTMTRDTSISTAYMELSRLRSDD
TAVYYCARGGWFDYWGQGTLVTVSS
Bimagrumab Variable Light Chain (VL)
(SEQ ID NO: 8)
QSALTQPASVSGSPGQSITISCTGTSSDVGSYNYVNWYQQHPGKA
PKLMIYGVSKRPSGVSNRFSGSKSGNTASLTISGLQAEDEADYYC
GTFAGGSYYGVFGGGTKLTVLGQ
Bimagrumab Heavy Chain (HC)
(SEQ ID NO: 9)
QVQLVQSGAEVKKPGASVKVSCKASGYTFTSSYINWVRQAPGQGL
EWMGTINPVSGSTSYAQKFQGRVTMTRDTSISTAYMELSRLRSDD
TAVYYCARGGWFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKRVEPKSCDKTHTCP
PCPAPEAAGGPSVELFPPKPKDTLMISRTPEVTCVVVDVSHEDPE
VKENWYVDGVEVHNAKTKPREEQYNSTYRVVSVLTVLHQDWLNGK
EYKCKVSNKALPAPIEKTISKAKGQPREPQVYTLPPSREEMTKNQ
VSLTCLVKGFYPSDIAVEWESNGQPENNYKTTPPVLDSDGSFFLY
SKLTVDKSRWQQGNVFSCSVMHEALHNHYTQKSLSLSPGK
Bimagrumab Light Chain (LC)
(SEQ ID NO: 10)
QSALTQPASVSGSPGQSITISCTGTSSDVGSYNYVNWYQQHPGKA
PKLMIYGVSKRPSGVSNRFSGSKSGNTASLTISGLQAEDEADYYC
GTFAGGSYYGVFGGGTKLTVLGQPKAAPSVTLFPPSSEELQANKA
TLVCLISDFYPGAVTVAWKADSSPVKAGVETTTPSKQSNNKYAAS
SYLSLTPEQWKSHRSYSCQVTHEGSTVEKTVAPTECS

In some embodiments, exemplary ActRII receptor antibodies of the disclosure comprise a variable heavy chain comprising the CDR amino acid sequences of SEQ ID NO: 1 (CDRH1), SEQ ID NO: 2 (CDRH2), and SEQ ID NO: 3 (CDRH3).

In some embodiments, exemplary ActRII receptor antibodies of the disclosure comprise a variable light chain comprising the CDR amino acid sequences of SEQ ID NO: 4 (CDRL1), SEQ ID NO: 5 (CDRL2), and SEQ ID NO: 6 (CDRL3).

In some embodiments, exemplary ActRII receptor antibodies of the disclosure comprise a variable heavy chain comprising the CDR amino acid sequences of SEQ ID NO: 1 (CDRH1), SEQ ID NO: 2 (CDRH2), and SEQ ID NO 3 (CDRH3); and comprise a variable light chain comprising the CDR amino acid sequences of SEQ ID NO: 4 (CDRL1), SEQ ID NO: 5 (CDRL2), and SEQ ID NO: 6 (CDRL3).

In some embodiments, exemplary ActRII receptor antibodies of the disclosure comprise a variable heavy chain comprising the amino acid sequence of SEQ ID NO: 7, or an amino acid sequence of at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or a variable light chain comprising the amino acid sequence of SEQ ID NO: 8, or an amino acid sequence of at least 90%, 91%, 92%, 93% 94%, 95%, 96%, 97%, 98%, or 99% sequence identity thereto.

In some embodiments, exemplary ActRII receptor antibodies of the disclosure comprise a heavy chain comprising the amino acid sequence of SEQ ID NO: 9, or an amino acid sequence of at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or a light chain comprising the amino acid sequence of SEQ ID NO: 10, or an amino acid sequence of at least 90%, 91%, 92%, 93%, 94%, 95%, 96%, 97%, 98%, or 99% sequence identity thereto.

In some embodiments, the ActRII receptor antibodies of the disclosure have an Fc domain, for example a human IgG1 Fc domain, a human IgG2 Fc domain, a human IgG3, Fc domain, or a human IgG4 Fc domain. In some embodiments, the Fc domain is wild type. In some embodiments, the Fc domain is modified, e.g. modified to increase serum half-life. In some embodiments, the Fc domain is a human IgG1 Fc domain, and modified to increase serum half-life. In some embodiments the modifications that increase serum half-life include one or more of 259I, 252Y, 307Q, 308F, 428L, 434H, 434F, 434Y, 434A, 434M, and 434S relative to a human IgG1 Fc domain according to the EU numbering scheme. In some embodiments, the modifications include one or more of 259I/434S, 308F/434S, 308F/428L/434S, 259I/308F/434S, 307Q/308F/434S, 250I/308F/434S, and 308F/1319L/434S relative to a human IgG1 Fc domain according to the EU numbering scheme.

In some embodiments, the ActRII receptor antibody binds to ActRIIB with a KD of 100 nM or less, 10 nM or less, 1 nM or less. Preferably, an ActRII receptor antibody brads to ActRIIB with an affinity of 100 pM or less (i.e. 100 pM, 50 pM, 10 pM, 1 pM or less). In some embodiments, the ActRII receptor antibody binds to ActRIIB with an affinity of between 10 and 20 pM.

In some embodiments, the ActRII receptor antibody binds to ActRIIB with a 5-fold greater affinity than to ActRIIA, more preferably 10-fold, still more preferably 50-fold, still more preferably 100-fold. In some embodiments, the ActRII receptor antibody binds to ActRIIA with an affinity of 100 pM or more (i.e. 250 pM, 500 pM, 1 nM, 5 nM or more).

### Other ActRII Pathway Agents

In other embodiments, the combination treatments described herein include an agent that binds an ActrRII receptor ligand, e.g., binds myostatin or activin directly. Such agents include, but are not limited to, a myostatin inhibitor (e.g., a myostatin antibody, or a myostatin small molecule antagonist), an activin inhibitor (e.g., an activin antibody, or an activin small molecule antagonist), or a soluble extracellular portion of ActRIIB or ActRIIA that can act as a “ligand sink” optionally further stabilized with an Fc. Accordingly, in some embodiments, provided herein are methods for treating a metabolic disorder of the disclosure, comprising administering to a subject in need a ActrII pathway agent (other than an ActRII receptor antibody) in combination with a GLP-1 agonist, wherein the agent is selected from the group consisting of a myostatin inhibitor, an activin inhibitor, or a soluble portion of an ActRII receptor.

In some embodiments, administration of both an ActRII pathway agent (other than an ActRII receptor antibody) and a GLP-1 agonist exhibits a beneficial effect, wherein an ActRII pathway agent (other than an ActRII receptor antibody) administered as part of a treatment with a GLP-1 agonist decreases the dosage of a GLP-1 agonist required to see an effect, and/or increases the therapeutic effect of the GLP-1 agonist, relative to the GLP-1 agonist alone. In some embodiments, a beneficial effect is demonstrated wherein a GLP-1 agonist decreases the dosage of the ActRII pathway agent (other than an ActRII receptor antibody) required to see an effect, and/or increases the therapeutic effect of the ActRII pathway agent, relative to administration of the ActRII pathway agent alone. In some embodiments the beneficial effect is increased safety of the combination relative to an ActRII pathway agent or a GLP-1 agent alone. In some embodiments the beneficial effect is increased efficacy of the combination relative to an ActRII pathway agent or a GLP-1 agent alone. In some embodiments the beneficial effect is increased tolerability relative to an ActRII pathway agent or a GLP-1 agent alone.

### III. GLP-1 Agonists

The glucagon-like peptide-1 receptor is a pancreatic beta cell receptor that stimulates insulin secretion upon binding of the hormone glucagon-like peptide 1 (GLP-1). GLP-1 is a type of incretin hormone, all of which regulate insulin release in response to blood sugar. GLP-1 agonists mimic the action of GLP-1 peptide and activate the GLP-1 receptor upon binding, stimulating insulin secretion.

However, GLP-1 agonists exhibit significant side effects and tolerability issues at effective doses. GLP-1 agonists have also been shown to decrease lean mass of a subject during treatment. Warnings and precautions for GLP-1 agonists (e.g., semaglutide) include but are not limited to: thyroid C-cell tumors, acute pancreatitis, acute gallbladder disease, hypoglycemia, acute kidney injury, severe adverse gastrointestinal reactions, hypersensitivity, e.g., anaphylactic reactions and angioedema, diabetic retinopathy complications in subjects Type 2 Diabetes, heart rate increase, and suicidal behavior and ideation. Adverse reactions reported in at least 5% of subjects treated with a GLP-1 agonist (e.g., semaglutide), include nausea, diarrhea, vomiting, constipation, abdominal pain, headache, fatigue, dyspepsia, dizziness, abdominal distension, eructation, hypoglycemia in patients with type 2 diabetes, flatulence, gastroenteritis, and gastroesophageal reflux disease. Thus, there is a need for optimized dosage and treatments with GLP-1 agonists.

As provided herein, the GLP-1 agonist of the disclosure may be a peptide, antibody, small molecule, or an aptamer. In some embodiments, the GLP-1 agonist is a peptide or peptide analog, including but not limited to: exenatide, exenatide extended-release, dulaglutide, liraglutide, lixisenatide, semaglutide, tirzepatide, cotadutide, noiiglutide, cotadutide, oxyntomodulin, or pemvidutide. In some embodiments, the GLP-1 agonist is a small molecule non-peptide agonist, e.g., danuglipron.

In some embodiments, the GLP-1 agonist is a dual agonist that binds and activates or deactivates a second receptor in addition to a GLP-1 receptor, wherein the second receptor is glucose-dependent insulinotropic (GIP) receptor or a glucagon (GCG) receptor. An exemplary dual acting GLP-1 agonist that binds both GLP-1 receptor and GIP receptor is tirzepatide. Exemplary dual acting GLP-1 agonists that binds both GLP-1 receptor and GCG receptor are cotadutide, noiiglutide, and oxyntomodulin, and are included in this disclosure as possible therapeutic agents to be used in combination with the ActRII receptor antibodies. In some embodiments, the GLP-1 agonist is also a GIP antagonist. In some embodiments, the GLP-1 agonist is also a GCG antagonist.

In some embodiments, the GLP-1 agonist is a triagonist of GIP/GLP-1/glucagon receptor, e.g., a GGG triagonist, e.g., LY343794.

### IV. Combination Therapies

A combination therapy, or a “combination” contemplated herein includes the co-administration of an ActRII receptor antibody (or other ActRII pathway agent) and a GLP-1 agonist, wherein the ActRII receptor antibody (or other ActRII pathway agent) may be administered prior to, after, or at the same time as the GLP-1 agonist. Such combination therapy may further include an initial loading dose of the ActRII receptor antibody (or other ActRII pathway agent). In exemplary embodiments, the co-administration includes administering an ActRII receptor antibody comprising the amino acid sequence of SEQ ID NOS: 1-6 and/or SEQ ID NOS: 7 and 8 (or a sequence with sequence identity of at least 90% thereto). In further exemplary embodiments, the co-administration includes administering an ActRII receptor antibody comprising the sequence of SEQ ID NOS: 1-6 and/or SEQ ID NOS: 7 and 8 (or a sequence with sequence identity of at least 90% thereto), and any of the GLP-1 agonists semaglutide, tirzepatide, or liraglutide.

The combination therapy of the disclosure comprising an ActRII receptor antibody and a GLP-1 agonist may be administered via any route. In some embodiments, the ActRII receptor antibody and/or the GLP-1 agonist is delivered orally, subcutaneously, intravenously, intranasally, transdermally, intraperitoneally, intramuscularly, intrapulmonary, vaginally, rectally, or intraocularly. In exemplary embodiments the ActRII receptor antibody is administered intravenously (IV) and/or subcutaneously, and the GLP-1 agonist is administered subcutaneously.

### Dosage and Timing of ActRII Receptor Antibodies in the Combination Therapies

In the combination therapies contemplated herein, in some embodiments, an ActRII receptor antibody is administered in a dose of about 3 mg/kg per the subject's body weight to about 50 mg/kg per the subject's body weight to a subject in need thereof.

In some embodiments, an ActRII receptor antibody is administered in a dose of about 3 mg/kg per the subject's body weight to about 50 mg/kg per the subject's body weight to a subject intravenously.

In some embodiments, an ActRII receptor antibody is administered to a subject in need thereof in a dose of about 3 mg/kg; in some embodiments in a dose of about 4 mg/kg; in some embodiments in a dose of about 5 mg/kg; in some embodiments in a dose of about 6 mg/kg; in some embodiments in a dose of about 7 mg/kg; in some embodiments in a dose of about 8 mg/kg; in some embodiments in a dose of about 9 mg/kg; in some embodiments in a dose of about 10 mg/kg; in some embodiments in a dose of about 11 mg/kg; in some embodiments in a dose of about 12 mg/kg; in some embodiments in a dose of about 13 mg/kg; in some embodiments in a dose of about 14 mg/kg; in some embodiments in a dose of about 15 mg/kg; in some embodiments in a dose of about 16 mg/kg; in some embodiments in a dose of about 17 mg/kg; in some embodiments in a dose of about 18 mg/kg; in some embodiments in a dose of about 19 mg/kg; in some embodiments in a dose of about 20 mg/kg; in some embodiments in a dose of about 21 mg/kg; in some embodiments in a dose of about 22 mg/kg; in some embodiments in a dose of about 23 mg/kg; in some embodiments in a dose of about 24 mg/kg; in some embodiments in a dose of about 25 mg/kg; in some embodiments in a dose of about 26 mg/kg; in some embodiments in a dose of about 27 mg/kg; in some embodiments in a dose of about 28 mg/kg; in some embodiments in a dose of about 29 mg/kg; in some embodiments in a dose of about 30 mg/kg; in some embodiments in a dose of about 31 mg/kg; in some embodiments in a dose of about 32 mg/kg; in some embodiments in a dose of about 33 mg/kg; in some embodiments in a dose of about 34 mg/kg; in some embodiments in a dose of about 35 mg/kg; in some embodiments in a dose of about 36 mg/kg; in some embodiments in a dose of about 37 mg/kg; in some embodiments in a dose of about 38 mg/kg; in some embodiments in a dose of about 39 mg/kg; in some embodiments, in a dose of about 40 mg/kg; in some embodiments in a dose of about 41 mg/kg; in some embodiments in a dose of about 42 mg/kg; in some embodiments in a dose of about 43 mg/kg; in some embodiments in a dose of about 44 mg/kg; in some embodiment in a dose of about 45 mg/kg; in some embodiments in a dose of about 46 mg/kg; in some embodiments in a dose of about 47 mg/kg; in some embodiments in a dose of about 48 mg/kg; in some embodiments in a dose of about 49 mg/kg; and in some embodiments in a dose of about 50 mg/kg.

In some embodiments the ActRII receptor antibody is administered to a subject in need thereof in a dose of 10 mg/kg. In some embodiments the ActRII receptor antibody is administered to a subject in need thereof in a dose of 30 mg/kg. In some embodiments the ActRII receptor antibody is administered to a subject in need thereof in a dose of 10 mg/kg intravenously. In some embodiments the ActRII receptor antibody is administered to a subject in need thereof in a dose of 30 mg/kg intravenously.

In some embodiments the ActRII receptor antibody is administered to a subject in need thereof two, three, four, or five or more times. In some embodiments, the ActRII receptor antibody is administered in a dosing regimen of once a week, twice a week, thrice a week, every two weeks, every four weeks, every six weeks, every twelve weeks, or every fifteen weeks. In some embodiments, the ActRII receptor antibody is administered every quarter of a year. In some embodiments, the ActRII receptor antibody is administered every 12 weeks in a dose of about 10 mg/kg. In some embodiments, the ActRII receptor antibody is administered every 12 weeks in a dose of 30 mg/kg.

In some embodiments, the ActRII receptor antibody is administered every 12 weeks in a dose of about 10 mg/kg intravenously. In some embodiments, the ActRII receptor antibody is administered every 12 weeks in a dose of 30 mg/kg intravenously.

In some embodiments an ActRII receptor antibody is administered in a dosing regimen of about 100 to about 600 mg, once, twice, three, or more times weekly. In some embodiments an ActRII receptor antibody is administered in a dosing regimen of about 200 to about 400 mg, for example, about 300 mg, once, twice, three, or more times weekly, or once every 2 weeks.

In some embodiments an ActRII receptor antibody is administered in a dosing regimen of about 200 to about 400 mg subcutaneously. In exemplary embodiments, an ActRII receptor antibody is administered in a dose of about 300 mg subcutaneously.

In some embodiments, the ActRII receptor antibody is administered in a dose of about 200 mg. In some embodiments, the ActRII receptor antibody is administered in a dose of about 200 mg once weekly. In some embodiments, the ActRII receptor antibody is administered in a dose of about 200 mg twice weekly. In some embodiments the ActRII receptor antibody is administered in a dose of 200 mg thrice weekly. In some embodiments, the ActRII receptor antibody is administered in a dose of about 200 mg every 2 weeks. In some embodiments, the ActRII receptor antibody is administered in a dose of about 200 mg subcutaneously.

In exemplary embodiments, the ActRII receptor antibody is administered in a dose of about 300 mg. In exemplary embodiments, the ActRII receptor antibody is administered in a dose of about 300 mg once weekly. In some embodiments, the ActRII receptor antibody is administered in a dose of about 300 mg twice weekly. In some embodiments the ActRII receptor antibody is administered in a dose of 300 mg thrice weekly. In some embodiments, the ActRII receptor antibody is administered in a dose of about 300 mg every 2 weeks. In exemplary embodiments, the ActRII receptor antibody is administered in a dose of about 300 mg subcutaneously. In exemplary embodiments, the ActRII receptor antibody is administered in a dose of about 300 mg once weekly subcutaneously.

In some embodiments, the ActRII receptor antibody is administered in a dose of about 400 mg. In some embodiments, the ActRII receptor antibody is administered in a dose of about 400 mg once weekly. In some embodiments, the ActRII receptor antibody is administered in a dose of about 400 mg twice weekly. In some embodiments the ActRII receptor antibody is administered in a dose of 400 mg thrice weekly. In some embodiments, the ActRII receptor antibody is administered in a dose of about 400 mg every 2 weeks. In some embodiments, the ActRII receptor antibody is administered in a dose of about 400 mg once weekly subcutaneously.

In some embodiments, the ActRII receptor antibody is administered in a dose of 600 mg. In some embodiments, the ActRII receptor antibody is administered in a dose of 600 mg once weekly, twice weekly, or thrice weekly, or every 2 weeks. In some embodiments, the ActRII receptor antibody is administered in a dose of about 600 mg subcutaneously.

In some embodiments the ActRII receptor antibody is administered in a dosing regimen of about 600 mg to about 3000 mg, for example, about 1000 mg once, twice, three or four times monthly. In some embodiments the ActRII receptor antibody is administered in a dosing regimen of about 600 mg to about 3000 mg subcutaneously. In some embodiments the ActRII receptor antibody is administered in a dosing regimen of about 600 mg to about 3000 mg, once, twice, three or four times monthly subcutaneously.

In some embodiments, the ActRII receptor antibody is administered in a dose of 750 mg. In some embodiments, the ActRII receptor antibody is administered in a dose of 750 mg once monthly. In some embodiments, the ActRII receptor antibody is administered in a dose of 900 mg. In some embodiments, the ActRII receptor antibody is administered in a dose of 900 mg once monthly. In some embodiments, the ActRII receptor antibody is administered in a dose of 1050 mg. In some embodiments, the ActRII receptor antibody is administered in a dose of 1050 mg once monthly.

In some embodiments, the ActRII receptor antibody is administered in a dose of 1200 mg. In some embodiments, the ActRII receptor antibody is administered in a dose of 1200 mg once monthly. In some embodiments, the ActRII receptor antibody is administered in a dose of 1350 mg. In some embodiments, the ActRII receptor antibody is administered in a dose of 1350 mg once monthly.

In some embodiments, the ActRII receptor antibody is administered in a dose of 1500 mg. In some embodiments, the ActRII receptor antibody is administered in a dose of 1500 mg once monthly. In some embodiments, the ActRII receptor antibody is administered in a dose of 1650 mg. In some embodiments, the ActRII receptor antibody is administered in a dose of 1650 mg once monthly. In some embodiments, the ActRII receptor antibody is administered in a dose of 1800 mg. In some embodiments, the ActRII receptor antibody is administered in a dose of 1800 mg once monthly.

In some embodiments, the ActRII receptor antibody is administered in a dose of 1950 mg. In some embodiments, the ActRII receptor antibody is administered in a dose of 1950 mg once monthly. In some embodiments, the ActRII receptor antibody is administered in a dose of 2100 mg. In some embodiments, the ActRII receptor antibody is administered in a dose of 2100 mg once monthly. In some embodiments, the ActRII receptor antibody is administered in a dose of 2250 mg. In some embodiments, the ActRII receptor antibody is administered in a dose of 2250 mg once monthly.

In some embodiments, the ActRII receptor antibody is administered in a dose of 2400 mg. In some embodiments, the ActRII receptor antibody is administered in a dose of 2400 mg once monthly. In some embodiments, the ActRII receptor antibody is administered in a dose of 2550 mg. In some embodiments, the ActRII receptor antibody is administered in a dose of 2550 mg once monthly. In some embodiments, the ActRII receptor antibody is administered in a dose of 2700 mg. In some embodiments, the ActRII receptor antibody is administered in a dose of 2700 mg once monthly. In some embodiments, the ActRII receptor antibody is administered in a dose of 2850 mg. In some embodiments, the ActRII receptor antibody is administered in a dose of 2850 mg once monthly. In some embodiments, the ActRII receptor antibody is administered in a dose of 3000 mg. In some embodiments, the ActRII receptor antibody is administered in a dose of 3000 mg once monthly.

### Loading Dose

In some embodiments, the administration of a loading dose of an ActRII receptor antibody in addition to the combination therapy may improve the outcome of the combination treatment. Without being bound by theory, it is thought that administration of a loading dose could maintain the level of the ActRII receptor antibody in the serum of the subject over time and further optimize the combination therapy. Accordingly, in some embodiments, a loading dose of an ActRII receptor antibody is administered.

In some embodiments, one or more loading doses of an ActRII receptor antibody are administered to a subject in need thereof at day 0 or week 0 prior to the start of the combination therapy. In some embodiments, one or more loading doses of an ActRII receptor antibody are provided 1 day, about 2 days, about 3 days, about 4 days, about 5 days, about 6 days, about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks before the combination therapy is administered. In exemplary embodiments, a loading dose is administered about 4 weeks before the start the combination therapy.

In some embodiments, a second administration of one or more loading doses are given after about 6 months, about 8 months, about 10 months, about 12 months, about 14 months, about 16 months, about 18 months, about 20 months, or about 24 months of administration of the combination therapy. In some embodiments, one or more loading doses are administered periodically or on a regular basis in addition to the combination therapy. In exemplary embodiments, a loading dose is administered first about 4 weeks prior to the start of the combination therapy, and administered about yearly thereafter.

In some embodiments, a subject is administered one or more loading doses followed by the combination therapy, and is then administered a second round of treatment, e.g. a second administration of one or more loading doses, followed by repeated combination therapy: likewise the treatment could include yet a third set of one or more loading doses, followed by a third round of the combination therapy, and so on and so forth.

In some embodiments, a loading dose is a dose of an ActRII receptor antibody the same concentration, a lower concentration, or a higher concentration as the dose of ActRII receptor antibody administered in the combination therapy. Accordingly, a loading dose may be about 3 mg/kg pet the subject's body weight to about 50 mg/kg per the subject's body weight, and all amounts in between. In some embodiments, a loading dose may be administered at about 3 mg/kg per the subject's body weight to about 50 mg/kg per the subject's body weight, intravenously.

In some embodiments, a loading dose of an ActRII receptor antibody is administered to a subject in need thereof in a dose of about 3 mg/kg; in some embodiments in a dose of about 4 mg/kg; in some embodiments in a dose of about 5 mg/kg; in some embodiments in a dose of about 6 mg/kg; in some embodiments in a dose of about 7 mg/kg; in some embodiments in a dose of about 8 mg/kg; in some embodiments in a dose of about 9 mg/kg; in some embodiments in a dose of about 10 mg/kg; in some embodiments in a dose of about 11 mg/kg; in some embodiments in a dose of about 12 mg/kg; in some embodiments in a dose of about 13 mg/kg; in some embodiments in a dose of about 14 mg/kg; in some embodiments in a dose of about 15 mg/kg; in some embodiments in a dose of about 16 mg/kg; in some embodiments in a dose of about 17 mg/kg; in some embodiments in a dose of about 18 mg/kg; in some embodiments in a dose of about 19 mg/kg; in some embodiments in a dose of about 20 mg/kg; in some embodiments in a dose of about 21 mg/kg; in some embodiments in a dose of about 22 mg/kg; in some embodiments in a dose of about 23 mg/kg; in some embodiments in a dose of about 24 mg/kg; in some embodiments in a dose of about 25 mg/kg; in some embodiments in a dose of about 26 mg/kg; in some embodiments in a dose of about 27 mg/kg; in some embodiments in a dose of about 28 mg/kg; in some embodiments in a dose of about 29 mg/kg; in some embodiments in a dose of about 30 mg/kg; in some embodiments in a dose of about 31 mg/kg; in some embodiments in a dose of about 32 mg/kg; in some embodiments in a dose of about 33 mg/kg; in some embodiments in a dose of about 34 mg/kg; in some embodiments in a dose of about 35 mg/kg; in some embodiments in a dose of about 36 mg/kg: in some embodiments in a dose of about 37 mg/kg; in some embodiments in a dose of about 38 mg/kg; in some embodiments in a dose of about 39 mg/kg; in some embodiments, in a dose of about 40 mg/kg: in some embodiments in a dose of about 41 mg/kg; in some embodiments in a dose of about 42 mg/kg; in some embodiments in a dose of about 43 mg/kg; in some embodiments in a dose of about 44 mg/kg; in some embodiment in a dose of about 45 mg/kg; in some embodiments in a dose of about 46 mg/kg; in some embodiments in a dose of about 47 mg/kg; in some embodiments in a dose of about 48 mg/kg; in some embodiments in a dose of about 49 mg/kg; and in some embodiments in a dose of about 50 mg/kg.

In exemplary embodiments, an ActRII receptor antibody loading dose is about 10 mg/kg per the subject's body weight. In other exemplary embodiments, an ActRII receptor antibody loading dose is about 30 mg/kg per the subject's body weight. In some embodiments, an ActRII receptor loading dose of about 10 mg/kg per the subject's body weight is administered subcutaneously. In some embodiments, an ActRII receptor loading dose of about 10 mg/kg per the subject's body weight is administered intravenously. In some embodiments, an ActRII receptor loading dose is about 30 mg/kg per the subject's both weight and is administered intravenously.

In some exemplary embodiments, an ActRII receptor antibody loading dose is about 200-400 mg per dose. In some exemplary embodiments, an ActRII receptor antibody loading dose is about 300 mg per dose. In some exemplary embodiments, an ActRII receptor antibody loading dose is about 200-400 mg per dose, and is administered subcutaneously.

### Dosage and Timing or GLP-1 Agonists in the Combination Therapies

In some embodiments of the combination therapies contemplated herein, a GLP-1 agonist is administered to a subject in need thereof in a dose of about 0.005 mg to about 3.0 mg, e.g., a dose of about 0.005 mg, about 0.01 mg, about 0.05 mg, about 0.1 mg, about 0.5 mg, about 1 mg, about 1.5 mg, about 2 mg, about 2.5 mg, or about 3.0 mg. In exemplary embodiments, the GLP-1 agonist is administered to a subject in need thereof in a dose of about 0.005 mg to about 3.0 mg subcutaneously.

In other embodiments of the combination therapies, a GLP-1 agonist administered to a subject in need thereof in a dose of about 1 to about 20 mg weekly, e.g., a dose of about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg. In exemplary embodiments, the GLP-1 agonist is administered to a subject in need thereof in a dose of about 5 mg, about 10 mg, or about 15 mg subcutaneously.

In some embodiments, the GLP-1 agonist is increased incrementally according to one of the following regimens: e.g., from about 0.2 mg to about 3.0 mg, from about 0.1 to about 1.0 mg, from about 0.25 mg to about 2.4 mg, or from about 0.25 mg to about 0.5 mg. In some embodiments the GLP-1 agonist is administered to a subject in need thereof in a dose of 0.5 mg. In some embodiments the GLP-1 agonist is administered to a subject in need thereof in a dose of 1.0 mg. In some embodiments the GLP-1 agonist is administered to a subject in need thereof in a dose of 1.7 mg. In some embodiments the GLP-1 agonist is administered to a subject in need thereof in a dose of 2.4 mg.

In some exemplary embodiments, the GLP-1 agonist is increased incrementally and is administered subcutaneously.

In some embodiments, a GLP-1 agonist is administered daily, every 2 days, every 3 days, every 4 days, every 5 days, every 6 days, every week, every 2 weeks, every 3 weeks, or every 4 weeks. In some embodiments the GLP-1 agonist is administered in a dose of 0.5 mg once weekly. In some embodiments the GLP-1 agonist is administered to a subject in need thereof in a dose of 1.0 mg once weekly. In some embodiments the GLP-1 agonist is administered to a subject in need thereof in a dose of 1.7 mg once weekly. In some embodiments the GLP-1 agonist is administered in a dose of 2.4 mg once weekly. In some embodiments, the GLP-1 agonist is administered weekly in a dose of about 0.25 mg from about week 0 to about week 4, and in a dose of about 0.5 mg at week 5 and weekly thereafter. In some embodiments the GLP-1 agonist dose is administered weekly in a dose of about 0.23 mg from about week 0 to about week 4, in a dose of about 0.5 mg from about week 5 to about week 8, in a dose of about 1.0 mg from about week 8 to about week 12, in a dose of about 1.7 mg from about week 12 to about week 15, in a dose of about 2.4 mg from about week 16 to about week 20, and a dose of about 2.4 mg from about week 20 and weekly thereafter.

In some embodiments, a GLP-1 agonist is administered daily, every 2 days, every 3 days, every 4 days, every 5 days, every 6 days, every week, every 2 weeks, every 3 weeks, or every 4 weeks. In some embodiments the GLP-1 agonist is administered in a dose of 5 mg once weekly. In some embodiments the GLP-1 agonist is administered m a dose of 10 mg once weekly. In some embodiments the GLP-1 agonist is administered in a dose of 15 mg once weekly.

### Combination Therapies—Exemplary Dosing Regimens

In some exemplary embodiments, the combination therapy of an ActRII receptor antibody and a GLP-1 agonist includes: a loading dose of an ActRII receptor antibody administered at week 0, followed by an ActRII receptor antibody in a dose of about 30 mg/kg administered intravenously 4 weeks after the loading dose, and every 12 weeks thereafter for the course of the treatment; and a GLP-1 agonist administered weekly and subcutaneously. In some embodiments, the GLP-1 agonist is administered weekly in a dose of about 2.4 mg or in a dose of about 15 mg. In some exemplary embodiments, the GLP-1 agonist of the combination therapy, e.g., semaglutide, is administered weekly in a dose of about 2.4 mg. In some embodiments, the GLP-1 agonist, e.g., semaglutide is administered weekly in a dose of about 0.5 mg. In some embodiments, the GLP-1 agonist, e.g., semaglutide is administered weekly in a dose of about 1.0 mg. In some embodiments, the GLP-1 agonist, e.g. semaglutide is administered weekly in a dose of about 2 mg. In other preferred embodiments, the GLP-1 agonist of the combination therapy, e.g., tirzepatide, is administered weekly in a dose of about 15 mg. In other embodiments, the GLP-1 agonist, e.g., tirzepatide, is administered weekly in a dose of about 5 mg. In other embodiments, the GLP-1 agonist, e.g., tirzepatide, is administered weekly in a dose of about 10 mg.

In other exemplary embodiments, the combination therapy of an ActRII receptor antibody and a GLP-1 agonist includes: an ActRII receptor antibody in a dose of about 300 mg administered once weekly, twice weekly, or once every two weeks and administered subcutaneously, and a GLP-1 agonist administered weekly and subcutaneously. In some exemplary embodiments the GLP-1 agonist is administered weekly in a dose of about 2.4 mg or about 15 mg. In some exemplary embodiments, the GLP-1 agonist of the combination therapy, e.g., semaglutide, is administered weekly in a dose of about 2.4 mg. In some embodiments, the GLP-1 agonist, e.g., semaglutide is administered weekly in a dose of about 0.5 mg. In some embodiments, the GLP-1 agonist, e.g., semaglutide is administered weekly in a dose of about 1.0 mg. In some embodiments, the GLP-1 agonist, e.g., semaglutide is administered weekly in a dose of about 2 mg. In other exemplary embodiments, the GLP-1 agonist of the combination therapy, e.g., tirzepatide, is administered weekly in a dose of about 15 mg. In other embodiments, the GLP-1 agonist, e.g., tirzepatide, is administered weekly in a dose of about 5 mg. In other embodiments, the GLP-1 agonist, e.g., tirzepatide, is administered weekly in a dose of about 10 mg.

### Combination Therapies—Timing

In some embodiments, the ActRII receptor antibody is administered at least 12 weeks prior, at least 10 weeks prior, at least 8 weeks prior, at least 6 weeks prior, at least 4 weeks prior, at least 2 weeks prior, at least 1 week prior, at least 1 day prior, or at least 1 hour prior to the administration of the GLP-1 agonist. The ActRII receptor antibody dosing regimen may further be preceded by the administration of a loading dose.

In some embodiments, the GLP-1 agonist is administered at least 2 weeks prior, at least 1 week prior, at least 5 days prior, at least 4 days prior, at least 3 days prior, at least 2 days prior, at least 1 day prior, at least 6 hours prior, or at least 1 hour prior to the antibody.

In some embodiments, the ActRII receptor antibody is administered in a dose of about 10 mg/kg every 12 weeks, optionally for 2 or more doses and the GLP-1 agonist is administered in a dose of about 0.5 mg to 2.4 mg every week. In some embodiments, the ActRII receptor antibody is administered in a dose of about 300 mg once a week, for 2 or more doses and the GLP-1 agonist is administered in a dose of about 0.5 mg to 2.4 mg every week. In some embodiments, administration of the GLP-1 agonist begins before the first administration of the ActRII receptor antibody. In some embodiments, administration of the GLP-1 agonist begins alter the first administration of the ActRII receptor antibody. The ActRII receptor antibody dosing regimen may further be preceded by the administration of a loading dose.

In some embodiments, the ActRII receptor antibody and the GLP-1 agonist are co-administered, i.e. administered at the same time. In some embodiments, the ActRII receptor antibody and the GLP-1 agonist are co-formulated and co-administered at the same time with a single injection. The ActRII receptor antibody dosing regimen may further be preceded by the administration of a loading dose.

### Subjects and Treatment

As put forth herein, the combination therapy is useful for the treatment of a metabolic disorder in a subject in need thereof. Metabolic disorders of the disclosure include but are not limited to obesity (e.g., glucocorticoid induced obesity), diabetes (Type I or Type II diabetes), metabolic syndrome, Prader-Willi syndrome, and hypertrophic lipodystrophy. In exemplary embodiments, the metabolic disorder is treated with an ActRII receptor antibody comprising the sequence of SEQ ID NOS: 1-6 and/or SEQ ID NOS: 7 and 8, or a sequence with sequence identity of at least 90% thereto, and a GLP-1 agonist selected from semaglutide, tirzepatide, and liraglutide.

In some embodiments, a subject in need of the combination treatments described herein is a subject who is overweight. In some embodiments, a subject in need of the combination treatments described herein includes a subject with a BMI of 30 or greater. In some embodiments, a subject in need of treatment includes a subject with a BMI of 27 or greater with an obesity related co-morbidity.

In some embodiments, a subject in need of treatment lacks glycemic control.

Subjects can be of any age, including youth. In some embodiments, the subject is over 20 years old, over 30 years old, over 40 years old, over 45 years old, over 50 years old, over 60 years old, or over 80 years old. In exemplary embodiments the subject is 45 or older. In some embodiments, the subject is 18 or older.

In exemplary embodiments, an ActRII receptor antibody is administered as part of a treatment program with a GLP-1 agonist. In some embodiments, administration of both an ActRII receptor antibody and a GLP-1 agonist treats one or more metabolic disorders, e.g., obesity and obesity related conditions. In some embodiments, the obesity related condition includes but is not limited to one or more of: glucose intolerance, prediabetes, insulin resistance, high triglycerides, overweight associated physical impairment, osteoporosis, renal disease, obstructive sleep apnea, sexual hormones impairment, endocrine reproductive disorders, osteoarthritis, gastrointestinal cancers, dyslipidaemia, hypertension, heart failure, coronary heart disease, stroke, gastroesophageal reflux disease and/or gallstones.

In some embodiments, administration of both an ActRII receptor antibody and a GLP-1 agonist improves glycemic control and/or treats Type II Diabetes.

In some embodiments, administration of both the ActRII receptor antibody and the GLP-1 agonist exhibits a beneficial effect, wherein an ActRII receptor antibody administered as part of a treatment with a GLP-1 agonist decreases the dosage of a GLP-1 agonist required to see an effect, and/or increases the therapeutic effect of the GLP-1 agonist, relative to the GLP-1 agonist alone. In some embodiments, a beneficial effect is demonstrated wherein a GLP-1 agonist decreases the dosage of the ActRII receptor antibody required to see an effect, and/or increases the therapeutic effect of the ActRII receptor antibody, relative to administration of the ActRII receptor antibody alone. In some embodiments the beneficial effect is increased safety of the combination of the ActRII receptor antibody and the GLP-1 agonist relative to either of an ActRII receptor antibody or a GLP-1 agent alone. In some embodiments the beneficial effect is increased efficacy of the combination of the ActRII receptor antibody and the GLP-1 agonist relative to either of an ActRII receptor antibody or a GLP-1 agent alone.

In some embodiments, the increased safety and/or tolerability of an agent is determined by 100%, 90%, 80%, 75%, 50%, or 25% lower risk of adverse reactions, side effects, number of warnings and precautions, or number of contraindications. In some embodiments, the level of the patient or physician reported side effects of the GLP-1 agonist are reduced at least 1.5×, 2×, 3×, 4×, 5×, or 10× when the GLP-1 agonist is administered as part of a treatment with an ActRII receptor antibody, relative to the GLP-1 agonist alone. In some embodiments the beneficial effect is increased efficacy of the combination of the ActRII receptor antibody and the GLP-1 agonist relative to either of an ActRII receptor antibody or a GLP-1 agent alone.

In some embodiments, a combination treatment of an ActRII receptor antibody and a GLP-1 agonist exhibits an additive therapeutic effect, wherein administration of both agents increases the efficacy of the treatment.

In some embodiments, a combination treatment of an ActRII receptor antibody and a GLP-1 agonist exhibits a synergistic therapeutic effect, wherein administration of both agents increases the efficacy more than the sum of the effect of each agent individually.

As provided herein, a combination treatment of an ActRII receptor antibody and a GLP-1 agonist reduces fat mass in a subject. In some embodiments, the treatment reduces fat mass in a subject (relative to pre-treatment) by at least 5%, at least 6%, at least 7%, at least 8%, at least 9%, at least 10%, at least 11%, at least 12%, at least 13%, at least 14%%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34% at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, over the treatment period. In some embodiments, the combined treatment of an ActRII receptor antibody and a GLP-1 agonist reduces fat mass in an additive or synergistic manner.

In some embodiments, a combination treatment of an ActRII receptor antibody and a GLP-1 agonist increases lean mass in the subject. In some embodiments, the treatment increases lean mass in a subject by at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34% at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, or at least 50% over the treatment period. In some embodiments, the combined treatment increases fat mass and decreases lean mass to a greater extent than a treatment with a GLP-1 agonist alone.

In some embodiments, a combination treatment of an ActRII receptor antibody and a GLP-1 agonist reduces fat mass and increases lean mass in a subject. In some embodiments, a combination treatment of an ActRII receptor antibody and a GLP-1 agonist reduces fat mass and increases lean mass in an additive or synergistic manner. In some embodiments, the treatment reduces fat mass in a subject by at least 5% (e.g., 5%-50%) and increases lean mass by at least 5% (e.g., 5%-50%) over the treatment period.

In some embodiments, a combination treatment of an ActRII receptor antibody and a GLP-1 agonist maintains lean mass in a subject. In some embodiments, a combined treatment of an ActRII receptor antibody and a GLP-1 agonist reduces fat mass and maintains lean mass in the subject in an additive or synergistic manner. In some embodiments, the combination treatment reduces fat mass in a subject by at least 5% (e.g., 5%-50%) and maintains lean mass over the treatment period.

In some embodiments, a combination treatment of an ActRII receptor antibody and a GLP-1 agonist reduces body weight in the subject. In some embodiments, the treatment reduces body weight in a subject by at least 5%, at least 10%, at least 20%, at least 30%, at least 40%, or at least 50% over the treatment period. In some embodiment, the combined treatment reduces body weight to a greater extent relative to treatment with either alone.

In some embodiments, a combination treatment of an ActRII receptor antibody and a GLP-1 agonist reduces the BMI of the subject. In some embodiments, the treatment reduces BMI in a subject by at least 5% at least 10%, at least 20%, at least 30%, at least 40%, or at least 50% over the treatment period. In some embodiment, the combined treatment reduces BMI to a greater extent relative to treatment with either alone.

In some embodiments, a combined treatment of an ActRII receptor antibody and a GLP-1 agonist reduces body weight in a synergistic manner. In some embodiments, a combined treatment of an ActRII receptor antibody and a GLP-1 agonist reduces central adiposity in the subject. In some embodiments, the treatment reduces central adiposity in a subject by at least 5% (e.g., 5%-50%) over the treatment period. In some embodiment, the combined treatment reduces central adiposity to a greater extent relative to treatment with either alone. In some embodiments, a combined treatment of an ActRII receptor antibody and a GLP-1 agonist reduces central adiposity in a synergistic manner.

In some embodiments, a combination treatment of an ActRII receptor antibody and a GLP-1 agonist reduces waist circumference in the subject. The average waist circumference of a woman in the United States is 38.7 inches, and the average waist circumference of a man is 40.2 inches, according to the United States Centers for Disease Control and Prevention (CDC). In some embodiments, the waist circumference may be decreased at least 5%, at least 10%, at least 20%, at least 30%, at least 40%, or at least 50% over the treatment period.

In some embodiments, fat mass in response to the combined treatment of an ActRII receptor antibody and a GLP-1 agonist is measured with BIA and/or DXA.

In some embodiments, a combined treatment of an ActRII receptor antibody and a GLP-1 agonist improves glycemic control in the subject. In some embodiments, the combined treatment improves glycemic control to a greater extent relative to treatment with either alone. In some embodiments, a combined treatment of an ActRII receptor antibody and a GLP-1 agonist improves glycemic control in a synergistic manner.

In some embodiments, glycemic control in response to the combined treatment of an ActRII receptor antibody and a GLP-1 agonist is measured by glucose and insulin levels, and the HOMA2 model is applied (www.dtu.ox.ac.uk/homacalculator/).

In some embodiments, the increased efficacy of the combination effect in a subject is determined by an at least 2-fold, at least 3-fold, at least 4-fold, at least 5-fold, at least 10-fold, or at least 20-fold improvement in at least one of the following measurements, relative to either treatment alone: body weight; bioelectrical impedance analysis (BIA) of lean mass and/or fat mass; waist to hip ratio; waist to height ratio; dual X-ray absorptiometry (DXA) of lean mass and/or fat mass; waist circumference; decreased BMI; blood lipids profile; leptin, adiponectin, and adipsin levels; IL-8 and/or IL-6 levels; urine biomarkers, hemoglobin A1c (HgbA1c) levels; hand dynamometry demonstrating muscle strength; glucose levels; insulin levels; short physical performance battery (SPPB); Impact of Weight on Quality of Life (IWQoL-Lice for CT) assessment; Short Form (36) Health Survey (SF-36) assessment; the homeostasis model assessment 2 (HOMA2); and physical activity monitoring via actigraphy.

### V. Pharmaceutical Combinations

In some embodiments, the ActRII receptor antibody and the GLP-1 agonist are combined in a pharmaceutical composition. In exemplary embodiments, the composition includes an ActRII receptor antibody comprising the sequence of SEQ ID NOS: 1-6 and/or SEQ ID NOS: 7 and 8 (or a sequence with sequence identity of at least 90% thereto), and the GLP-1 agonist semaglutide. In other exemplary embodiments, the composition includes an ActRII receptor antibody comprising the sequence of SEQ ID NOS: 1-6 and/or SEQ ID NOS: 7 and 8 (or a sequence with sequence identity of at least W % thereto), and the GLP-1 agonist tirzepatide.

In some embodiments the composition includes an excipient, or carrier, e.g., an aqueous carrier. A variety of aqueous carriers can be used, e.g., buffered saline. The compositions may contain pharmaceutically acceptable auxiliary substances as those required to approximate physiological conditions such as pH and buffering agents, toxicity countering agents, e.g., disodium phosphate dihydrate, monosodium phosphate, sodium acetate, sodium chloride, potassium chloride, calcium chloride, hydrochloric acid, sodium hydroxide, L-histidine, L-histidine hydrochloride, and sodium lactate. The concentration of active agent in these formulations can vary and are selected based on fluid volumes, viscosities, and body weight in accordance with the particular mode of administration selected and the patient's needs (e.g., Remington's Pharmaceutical Science (15th ed., 1980) and Goodman & Gillman. The Pharmacological Basis of Therapeutics (Hardman et al., eds., 1996)). The compositions may contain pharmaceutically acceptable auxiliary substances such as those that contribute to the stability and activity of the pharmacologically active agent or agents, including but not limited to trehalose, sucrose, or other sugars and polysorbate 20, polysorbate 60, polysorbate 80 or other emulsifiers or stabilizers.

### EXAMPLES

### Example 1: Comparison of the Safety and Efficacy of Bimagrumab with Semaglutide in Adults Who are Overweight or Obese

A randomized, placebo-controlled study was designed to compare the safety and efficacy of bimagrumab with that of open label semaglutide in adults 45 and older who are overweight or obese. The purpose of the study is to compare the effect of bimagrumab and semaglutide on fat loss, when given in combination or singly, in adults ages 45 and above who are obese or who are overweight with at least one obesity related co-morbidity.

### Primary Objective

To assess if the combination of bimagrumab and semaglutide is superior to either therapy administered alone with respect to fat mass loss as measured by change in waist circumference (cm) from baseline at Week 0 to Week 24. The study may also be extended up to Week 28, Week 48, Week 68, or Week 72.

### Secondary Objectives

I. To assess the safety and tolerability of bimagrumab and semaglutide when administered in combination and when administered as monotherapy.
II. To evaluate the effect of the combination of bimagrumab and semaglutide compared to each therapy administered alone with respect to change in lean mass (kg and % change) from baseline at Week 0 to Week 24, as measured by DXA.
III. To assess if the combination of bimagrumab and semaglutide is superior to either therapy administered alone with respect to the change in muscle strength as measured by hand dynamometry from baseline at Week 0 to Week 24.
IV. To evaluate the effect of the combination of bimagrumab and semaglutide compared to each therapy administered alone with respect to change in fat mass (kg and % change) from baseline at Week 0 to Week 24, as measured by DXA.
V. To assess change from baseline at Week 0 to Week 24 in body weight (kg) and body composition as measured by bioelectrical impedance analysis (BIA) for each treatment group.
VI. To assess the proportion of participants in each treatment group experiencing ≥5% body weight reduction from baseline at week 0 to Week 24 (%).
VII. To assess change from baseline at Week 0 to Week 24 in systolic and diastolic blood pressure (mmHg) for each treatment group.
VIII. To assess change from baseline at Week 0 to Week 24 in HbA1C and HOMA2 for each treatment group
IX. To determine the change of all the objective primary and secondary endpoints above from week 24 to week 36
X. To assess change from baseline at Week 0 to Week 24 in participants health status (SF-36)
XI. To assess change from baseline at Week 0 to Week 24 in participants quality of life (IWQoL-Lite for CT)

### Efficacy Endpoints

To determine the efficacy of the combination of bimagrumab and semaglutide with respect to fat mass loss, the following markers are assessed: waist circumference, blood pressure; fat mass by DXA; lean mass by DXA; body weight; BMI; lipids; HgbA1c levels; glucose levels; insulin levels: short physical performance battery (SPPB); physical activity monitoring via actigraphy; exploratory urine biomarkers; leptin, adiponectin, adipsin, IL-18, and IL-6 as adipose tissue biomarkers, and Activin A as a pharmacodynamic (PD) biomarker.

### Safety Endpoints

To determine the safety of the treatment, clinically determined adverse events (AEs) are monitored. These include but are not limited to treatment emergent adverse events (TEAEs) and serious adverse events (SAEs). Further, the change from baseline of patient health markers from week 0 to week 24 are monitored, including: heart rate (bpm), amylase (U/L), lipase (U/L), Alkaline Phosphatase ALP (U/L), aspartate aminotransferase AST (U/L), and alanine aminotransferase ALT (U/L).

### Study Design

The study is randomized, partially blinded, and placebo-controlled, with a 9-arm, multi-center design. Participants are randomized to one of the following treatment arms:
a) Bimagrumab 30 mg/kg
b) Bimagrumab placebo
c) Low dose semaglutide (0.5 mg/1.0 mg)+bimagrumab 30 mg/kg
d) High dose semaglutide (2.4 mg)+bimagrumab 30 mg/kg
e) Low dose semaglutide (0.5 mg/1.0 mg)+bimagrumab placebo
f) High dose semaglutide (2.4 mg)+bimagrumab placebo
g) Bimagrumab 10 mg/kg
h) Low dose semaglutide (0.5 mg/1.0 mg)+bimagrumab 10 mg/kg
i) High dose semaglutide (2.4 mg)+bimagrumab 10 mg/kg

While bimagrumab and bimagrumab placebo treatment are kept blinded, semaglutide treatment is open-label. The study consists of a screening visit to assess eligibility, followed by visits and phone calls every 6 weeks during the treatment phase, until the follow up at week 24. The End of Study visit is performed at week 36.

### Investigational Product, Dosage and Mode of Administration

As shown above in the study design, bimagrumab is given in a dose of either 10 mg/kg or 30 mg/kg each dose by intravenous (IV) infusion, for a total number of 2 doses, every 12 weeks. The rationale for a 24 week exposure duration and primary endpoint assessment is that this duration allows time for observation of differential effects from the two bimagrumab doses and the two semaglutide doses relative to each other and to the placebo.

### Semaglutide Weekly Subcutaneous Injection

The dose of semaglutide is incrementally increased from 0.25 mg subcutaneous weekly to 2.4 mg subcutaneous weekly according to the label instructions for the high dose group or from 0.25 mg to 0.5 mg subcutaneous weekly for the low dose group. The control arm for semaglutide is a no-treatment group and is not given a subcutaneous injection.

### Reference Therapy, Dosage and Mode of Administration

The bimagrumab placebo includes dextrose 5% in water (D5W) and is administered by IV infusion in 2 doses, one dose at week 0 and the second dose at week 12.

### Duration of Study

The study is up to 40 weeks in duration, including a 4-week screening period, a 24-week treatment period, and a 12 week follow up period.

### Inclusion Criteria

1. A written informed consent must be obtained before any study-related assessment.
2. Men aged ≥18, women aged ≥40.
3. BMI ≥30 or BMI ≥27 with one or more obesity-associated comorbidities.
4. Have a stable body weight (±3 kg) within three months of screening
5. Have a history of at least 1 self-reported unsuccessful dietary effort to lose body weight
6. Able to communicate well with the investigator, comply with the study requirements and adhere to the diet and activity programs for the study duration.

### Exclusion Criteria

1. History of a severe reaction to a monoclonal antibody therapy
2. Use of other investigational drugs at the time of enrollment or within 30 days or 5 half-lives of enrollment, whichever is longer, or longer if required by any other limitation of participation in an investigational trial based on local regulations.
3. Prior serious hypersensitivity reaction to semaglutide or any of the excipients in Ozempic or Wegovy.
4. Patient weight>150 kg
5. History of clinically significant condition, for example cardiovascular or pulmonary disease or osteoarthritis, that precludes regular walking for exercise
6. Contraindication to following a 500 calorie daily deficit, high protein diet
7. Pregnant or nursing women.
8. Women of child-bearing potential, defined as all women physiologically capable of becoming pregnant. UNLESS they are using highly effective methods of contraception during dosing and for 6 months after the last dose of bimagrumab.

### Highly Effective Contraception Methods Include:

i. Total abstinence. Periodic abstinence (e.g., calendar, ovulation, symptothermal, post-ovulation methods) and withdrawal are not acceptable methods of contraception.
ii. Female sterilization (surgical bilateral oophorectomy with or without hysterectomy) or tubal ligation at least 6 weeks before taking study treatment. In case of oophorectomy alone, only when the reproductive status of the woman has been confirmed by follow up hormone level assessment.
iii. Male sterilization (at least 6 months prior to screening). For female participants in the study, the vasectomized male partner should be the sole partner for that participant.
iv. Combination of any two of the following methods (a+b or a+c or b+c):
   a) Oral, injected or implanted hormonal methods of contraception or other forms of hormonal contraception with comparable efficacy (failure rate<1%), e.g., hormone vaginal ring or transdermal hormone contraception.
   b) Intrauterine device or intrauterine system.
   c) Barrier methods of contraception e.g., condom or occlusive cap (diaphragm or cervical/vault caps) with spermicidal foam/gel/film/cream/vaginal suppository.

In case of use of oral contraception women should have been stable on the same pill for a minimum of 3 months before taking study treatment.

Women are considered post-menopausal and not of childbearing potential if they hay a had 12 months of natural (spontaneous) amenorrhea with an appropriate clinical profile (e.g., age appropriate, history of vasomotor symptoms) or have had surgical bilateral oophorectomy (with or without hysterectomy) or tubal ligation at least six weeks ago. In the case of oophorectomy alone, only when the reproductive status of the woman has been confirmed by follow up hormone level assessment is the woman considered not of childbearing potential.
9. History of malignancy of any organ system, treated or untreated within the past five years, regardless of whether there was evidence of local recurrence or metastases, except non-melanoma skin cancer treated with local therapy, prostate cancer managed with watchful waiting, or breast or cervical carcinoma treated with local excision alone.
10. Personal or family history of medullary thyroid carcinoma or in patients with Multiple Endocrine Neoplasia syndrome type 2.
11. Disease other than cancer known to cause cachexia or muscle atrophy, disease known to cause GI malabsorption disease associated with lipodystrophy.
12. Diagnosis of diabetes plus current use of any anti-diabetic drug. Metabolic syndrome is not an exclusion, even if managed with an anti-diabetic drug such as metformin or an SGLT2 inhibitor.
13. Uncontrolled hypothyroidism. Hypothyroid patients treated with hormone replacement therapy must be on a stable dose for at least 6 weeks poor to the Screening visit.
14. Significant psychiatric disorder, clinically manifest peripheral vascular disease or disorder, or systemic disorder which could affect any of the efficacy assessments (e.g., diabetic neuropathy, chronic fatigue syndrome, schizophrenia, bipolar disorder, severe depression, intermittent claudication).
15. Known heart failure classified as New York Heart Association Class III and IV or a history of chronic hypotension (systolic blood pressure<100 mmHg).
16. Systolic blood pressure >180 or <90 mm Hg or diastolic blood pressure >100 or <50 mmHg at screening, or malignant hypertension.
17. ECG showing clinically significant abnormalities including any current supra-ventricular arrhythmia with an uncontrolled ventricular response (mean heart rate>100 beats per minute [bpm]) at rest despite medical or deice therapy, or any history of spontaneous or induced sustained ventricular tachycardia (heart rate>100 bpm for 30 sec) despite medical or device therapy, or any history of resuscitated cardiac arrest or presence of an automated internal cardioverter-defibrillator.
18. History of unstable angina, myocardial infarction, coronary artery bypass graft surgery, or percutaneous coronary intervention (such as angioplasty or stent placement) within 180 days of the Screening visit.
19. Prolonged QT syndrome or QTcF>450 msec (Fridericia Correction) for males and >470 msec for females at screening or baseline at repeated assessment.
20. Significant coagulopathy, platelet count less than 75,000/mm³, hemoglobin less than 11.0 g/dL.
21. Liver disease or liver injury as indicated by abnormal liver function tests such as SGOT (AST), SGPT (ALT), alkaline phosphatase, or serum bilirubin (except Gilbert's Disease). The Investigator should be guided by the following criteria:

Any single transaminase may not exceed 3× times upper limit of normal (ULN). A single parameter elevated up to and including 3×ULN should be re-checked as soon as possible, and in all cases, at least prior to randomization, to rule out any lab error.

If the total bilirubin concentration is increased above 1.5×ULN, total bilirubin should be differentiated into the direct and indirect reacting bilirubin. In any case, serum bilirubin should not exceed the value of 1.6 mg/dL (27 μmol/L).
22. Known history or presence of severe acute or chronic liver disease (compensated or decompensated), known gallbladder or bile duct disease, acute or chronic pancreatitis, acute renal failure or chronic renal failure stage 3 or worse.
23. History of hepatitis B or HIV. History of hepatitis A or hepatitis C successfully treated is not exclusionary.
24. Use of any prescription drugs known to affect muscle mass adversely, including anti-androgens (such as luteinizing hormone releasing hormone (LHRH) agonists), anti-estrogens (tamoxifen, etc.), etc. Low dose estrogen replacement therapy in post-menopausal women is acceptable and 5-alpha reductase inhibitors in men are acceptable.
25. Lack of peripheral venous access.
26. Donation or loss of 400 mL or more of blood within eight weeks prior to initial dosing, or longer if required by local regulation, or plasma donation (>250 mL) within 14 days prior to the first dose.
27. Acute illness within the 30 days prior to the Screening visit that, in the opinion of the investigator, affects lower extremity function or the patient's ability to participate in the study.
28. Smoking more than one pack of cigarettes daily.
29. Using Cannabis more than twice weekly.
30. Drinking 5 or more alcoholic beverages on the same occasion on each of 5 or more days in the past 30 days.

### Example 2: Comparison of the Safety and Efficacy of Bimagrumab with Semaglutide in Adults Who are Overweight or Obese

A study as described in Example 1, to compare the safety and efficacy of bimagrumab with that of open label semaglutide in adults 45 and older who are overweight or obese, may be modified as described herein. Unlike Example 2, this dosing paradigm includes the administration of a loading dose.

### Secondary Objectives

The secondary objectives noted in Example 1 are incorporated herein, additional secondary objectives include:
XII. To evaluate the effect of the combination of bimagrumab and semaglutide compared to each therapy administered alone using a change in bioelectrical impedance (percent Ohms change) from the baseline at Week 0 to Week 24.
XIII. To determine the change in all primary and secondary objectives from week 24 to week 36.

### Efficacy Endpoints

The efficacy endpoints noted in Example 1 are incorporated herein, additional efficacy endpoints include: lipid levels (total cholesterol (TC), high-density lipoprotein (HDL), low-density lipoprotein (LDL), and triglycerides (TG)), urine mibroalbumin levels, the Impact of Weight on Quality of Life (IWQoL-Lite for CT) assessment, and Short Form (36) Health Survey (SF-36) assessment.

### Study Design

The study comprises visits and phone calls every 4 weeks during the treatment phase.

### Investigational Product, Dosage and Mode of Administration

Bimagrumab is given in a dose of either 10 mg/kg (low dose) or 30 mg/kg (high dose) each by intravenous (IV) infusion, at week 0 (loading dose), week 4, and week 16. This dosing regimen is every 12 weeks with the inclusion of a loading dose at week 0 prior to the start of the dosing regimen at week 4.

### Semaglutide Weekly Subcutaneous Injection

As described in Example 1, the dose of semaglutide is incrementally increased from 0.25 mg subcutaneous weekly to 2.4 mg subcutaneous weekly according to the label instructions for the high dose group or from 0.25 mg to 0.5 mg subcutaneous weekly for the low dose group.

The dose escalation of semaglutide for the low dose group may be as follows: 0.25 mg from week 0 to week 4, and 0.5 mg thereafter.

The dose escalation of semaglutide for the high dose group is as follows: in a dose of 0.25 mg weekly from week 0 to week 4, in a dose of 0.5 mg weekly from week 5 to week 8, in a dose of 1.0 mg weekly from week 8 to week 12, in a dose of 1.7 mg weekly from week 12 to week 15, in a dose of 2.4 mg weekly from week 16 to week 20, and a dose of 2.4 mg weekly from week 20 to week 24. Patients who are unable to tolerate a weekly dose of 0.5 mg or higher may drop down to the preceding lower dose.

### Exclusion Criteria

The exclusion criteria noted in Example 1 are incorporated herein, additional exclusion criterion of study participants includes a history of familial hypertriglyceridemia or history of fasting triglyceride greater than 500 mg/dl (5.65 mmol/L).

### Example 3: Effects of the Combination Treatment of an ActRII Receptor Antibody and a Glucagon-Like Peptide-1 Receptor (GLP-1) on Fat Mass and Lean Mass in Obese Mice: Initial Study

In overweight and obese patients, the ActRII receptor antibody bimagrumab and the glucagon-like peptide-1 receptor (GLP-1) semaglutide each were shown to induce greater than 20% body fat loss over approximately 1 year of therapy. However, bimagrumab caused a 4-8% increase in muscle mass while semaglutide caused lean mass loss. Mouse experiments were designed to determine the efficacy of the combination of bimagrumab and semaglutide with respect to fat loss and determine to what extent the bimagrumab could attenuate the lean mass loss that accompanied semaglutide therapy. The present disclosure describes the surprising result that a combination treatment of an ActRII receptor antibody, bimagrumab and a glucagon-like peptide-1 receptor (GLP-1), semaglutide, reduced fat mass to a greater extent than an ActRII receptor antibody alone, while also increasing lean mass, in obese mice.

### Methods

C57B1/6J mice at 5-6 weeks of age were fed a 60% fat diet for 14 weeks and reached an average body weight of 49 grams at the start of dosing, as an obese mouse model. Employing a modified 3×3 factorial design, groups of mice (n=8 per group) received a murinized version of bimagrumab, CDD866, at 0.3 mg/kg or 20 mg/kg intraperitoneally (i.p.) weekly and/or semaglutide at 5 μg/kg or 40 μg/kg subcutaneously (s.c.) daily. The high fat diet was continued through the treatment period, and food intake and body weight were monitored twice weekly. Clinical observations, body composition by MRI, and physical activity, were assessed once weekly.

### Results

Mice treated with semaglutide alone or together with CDD866 had an initial decrease in food intake that recovered to baseline or above by 2 weeks, and at that time there were no clinical observations of concern in any animal. Indeed, the animals receiving the high dose of both drugs trended towards being more active than the vehicle-alone groups. In the mice that received a dose of 20 mg/kg CDD866 alone, lean mass increased 13%. For the same 20 mg/kg dose of CDD866 in combination with a semaglutide dose of 40 μg/kg, lean mass increased by 5%, compared to a lean mass decrease of 4% with semaglutide 40 ug/kg alone. Fat mass after 2 weeks of treatment decreased by 8% for a 20 mg/kg dose of CDD866 and by 16% for a 40 μg/kg dose of semaglutide alone. Notably, fat mass decreased by 31% in the mice that received 20 mg/kg of CDD866 in combination with 40 μg/kg semaglutide. Bimagrumab and semaglutide when administered together have at least an additive efficacy with respect to fat loss in obese mice while simultaneously yielding a lean mass increase.

### Example 4: Pharmacokinetic Effects of the Combination of the ActRII Receptor Antibody CDD866 and the Glucagon-Like Peptide-1 Receptor (GLP-1) Semaglutide

A study was designed to determine the effect of CDD866 on semaglutide pharmacokinetics (PK), as well as the effect of semaglutide on CDD866 PK. Notably, each of CDD866 and semaglutide demonstrated minimal effects on the other's overall exposure as assessed by AUC.

### Methods

Dedicated cohorts of diet induced obesity (DIO) mice were used. Blood sampling was performed at 5 time points, with 3 mice per time point. Table 1 shows the effects of CDD866, administered in a dose of 20 mg/kg, on semaglutide PK, administered in a dose of 40 μg/kg.

TABLE 1
Effects of CDD866 on semaglutide PK

| | Analyte | | |
|---|---|---|---|
| | Semaglutide | | |
| | Group | | |
| | Semaglutide- | Semaglutide- | CDD866-20 mpk + |
| | 5 ug/kg | 40 ug/kg | Semaglutide-40 ug/kg |
| | Matrix | | |
| | Mean Plasma | Mean Plasma | Mean Plasma |
| Dosage (mg/kg) | 0.00500 | 0.0400 | 0.0400 |
| Cₘₐₓ (ng/mL) | 3.60 | 31.7 | 20.2 |
| Tₘₐₓ (h) | 4.00 | 4.00 | 2.00 |
| T_{1/2} (h) | ND | 9.33 | 10.6 |
| Tₗₐₛₜ (h) | 8.00 | 24.0 | 24.0 |
| AUC₀₋ₗₐₛₜ (ng · h/mL) | 23.1 | 425 | 225 |
| AUC₀₋₂₄ (ng · h/mL) | ND | 425 | 225 |
| AUC_{0-inf} (ng · h/mL) | ND | 524 | 293 |
| MRT₀₋ₗₐₛₜ (h) | 4.38 | 10.0 | 9.76 |
| MRT_{0-inf} (h) | ND | 15.2 | 16.6 |

Table 2, below, shows the effects of semaglutide, administered in a dose of 40 μg/kg on CDD866 PK, administered in a dose of 20 mg/kg.

TABLE 2
Effects of semaglutide on CDD866 PK

| | Analyte | | |
|---|---|---|---|
| | CDD866 | | |
| | Group | | |
| | | | CDD866-20 mpk + |
| | CDD866-3 mpk | CDD866-20 mpk | Semaglutide-40 ug/kg |
| | Martix | | |
| | Plasma | Plasma | Plasma |
| Dosage (mg/kg) | 3.00 | 20.0 | 20.0 |
| Cₘₐₓ (ng/mL) | 34933 | 256333 | 315000 |
| Tₘₐₓ (h) | 24.0 | 24.0 | 4.00 |
| T_{1/2} (h) | 31.4 | 153 | 93.8 |
| Tₗₐₛₜ (h) | 168 | 168 | 96.0 |
| AUC₀₋₂₄ (ng · h/mL) | 419196 | 3075996 | 7026348 |
| AUC₀₋ₗₐₛₜ (ng · h/mL) | 2504837 | 27075590 | 22551850 |
| AUC_{0-inf} (ng · h/mL) | 2592003 | 57420480 | 45961460 |
| MRT₀₋ₗₐₛₜ (h) | 61.1 | 78.4 | 43.0 |
| MRT_{0-inf} (h) | 66.2 | 243 | 139 |

### Example 5: Effects of the Combination Treatment of an ActRII Receptor Antibody, CDDS66, and a GLP-1 Agonist, Semaglutide, on Fat Mass and Lean Mass in Mice: Continued Study

The present disclosure provides the surprising result that the combination of an ActRII receptor antibody and a GLP-1 agonist reduced fat mass synergistically, while also maintaining or increasing lean mass.

As described above in Example 3, mouse experiments were designed to determine the efficacy of the combination of an ActRII receptor antibody and a GLP-1 agonist with respect to fat loss, and to determine to what extent an ActRII receptor antibody could attenuate the lean mass loss that accompanies GLP-1 agonist treatment. As shown in Table 3, below, the combination of the ActRII receptor antibody CDD866 and the GLP-1 agonist, semaglutide, reduced fat mass synergistically in DIO mice, and increased lean mass relative to the control.

TABLE 3
Effects of the combination of CDD866 and semaglutide by day 22

| Day 22 | Body weight (g) | Fat (g) | Lean mass(g) |
|---|---|---|---|
| Vehicle | 56.8 ± 0.7 | 25.6 ± 0.3 | 27.9 ± 0.7 |
| CDD866 (20 mg/kg) | 57.8 ± 1.0 | 23.9 ± 0.6 | 31.9 ± 0.5 |
| Semaglutide (40 μg/kg) | 45.35 ± 1.1 | 17.8 ± 0.5 | 25.8 ± 1.0 |
| CDD866 + Semaglutide | 43.89 ± 1.4 | 12.1 ± 1.6 | 29.7 ± 0.6 |

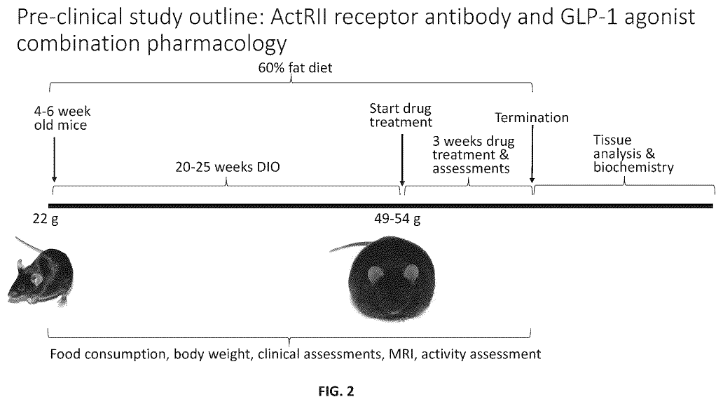

As described in the study outline in FIG. 2, efficacy measurements of the combination of the ActRII receptor antibody and GLP-1 agonist included food consumption, body weight, clinical biomarkers, MRI, and physical activity levels m DIO mice during the 3 weeks of treatment.

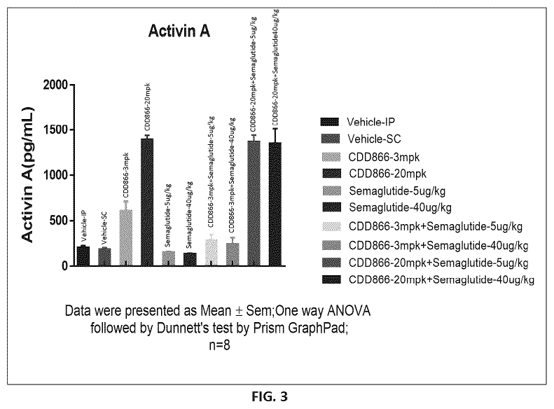

Pharmacodynamics (PD) of the ActRII receptor antibody were assessed in the presence of semaglutide. As shown in FIG. 3, there was little effect of the GLP-1 agonist, semaglutide, on the CDD866 regulation of circulating Activin A levels in the blood. These results demonstrate that GLP-1 agonists do not interfere with ActRII receptor antibody function.

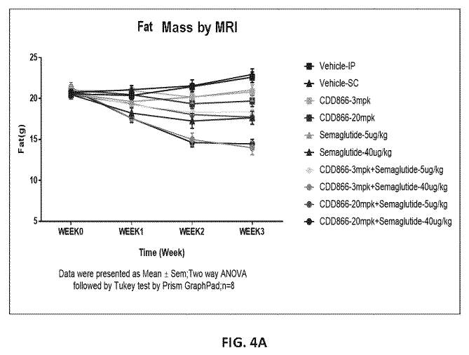

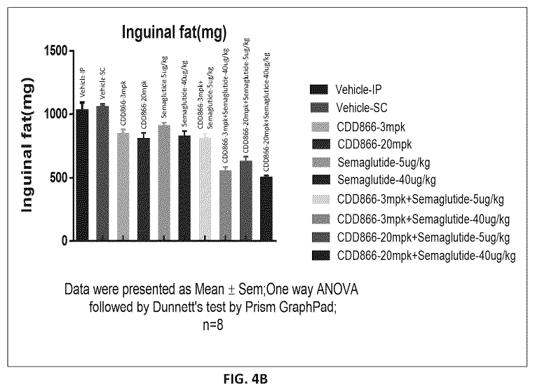

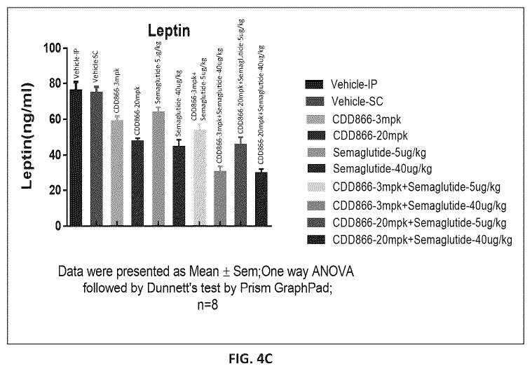

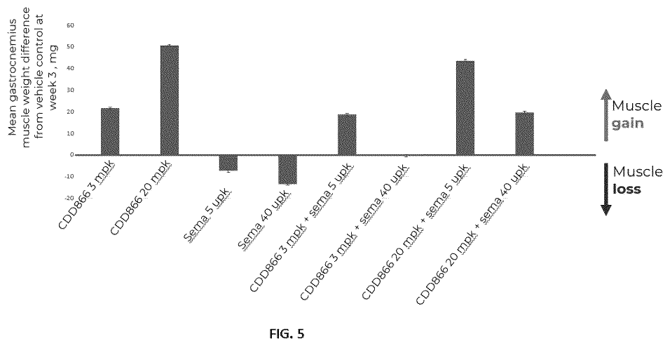

Notably. CDD866 in combination with semaglutide, administered to DIO mice, demonstrated at least an additive effect on inguinal fat mass, and leptin levels, and a synergistic effect on fat mass loss (FIGS. 4A-4C, Table 3). Further, CDD866 in combination with semaglutide reversed the lean mass muscle loss of semaglutide alone (FIG. 5). These results demonstrate that the combination of CDD866 and the GLP-1 agonist semaglutide provides both a synergistic reduction of fat mass loss, and also serves to counter the decrease in lean mass exhibited by GLP-1 agonists alone.

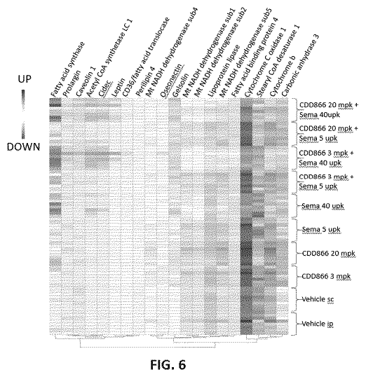

RNAseq of inguinal fat (issue RNA transcripts was also performed. The RNAseq transcript results are shown in FIG. 6, and are consistent with a reduction in fat mass in adipocytes via lipid mobilization signaling. The RNA library for the RNAseq experiments contained about 44-73 million reads per sample, and the 20 genes with the greatest changes in transcript levels in response to the CDD866 and semaglutide combination are shown in FIG. 6. These results demonstrate that the combination of CDD866 and semaglutide activates lipid mobilization and decreases new lipid storage in fat tissue.

### Example 6: Effects of the Combination Treatment of an ActRII Receptor Antibody, CDD866, and Additional GLP-1 Agonists on Fat Mass and Lean Mass in Mice

To determine whether other GLP-1 agonists could similarly function to synergistically reduce tat mass with an ActRII receptor antagonist, the GLP-1 semaglutide, liraglutide, and tirzepatide were tested in combination with CDD866. The combination of the ActRII receptor antagonist CDD866 and the GLP-1 agonist tirzepatide resulted in an even greater synergistic reduction in fat mass loss relative to that of semaglutide, while still increasing lean mass relative to the control.

### Methods

DIO mice received a murinized version of bimagrumab, CDD866, at 20 mg/kg intraperitoneally (i.p.) weekly in combination with semaglutide (40 μg/kg once daily), liraglutide (40 μg/kg twice daily), or tirzepatide at 45 μg/kg once daily and after day 11, 22.5 μg/kg once daily) subcutaneously (s.c.). The GLP-1 agonists in this study were not dose escalated. The tirzepatide dose was dropped to 22.5 μg/kg on day 11 to moderate the observed drop in food intake.

### Results

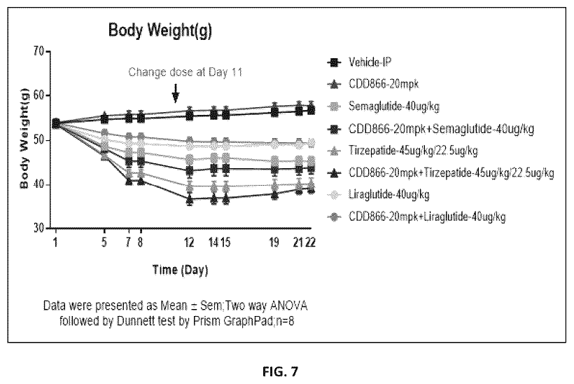

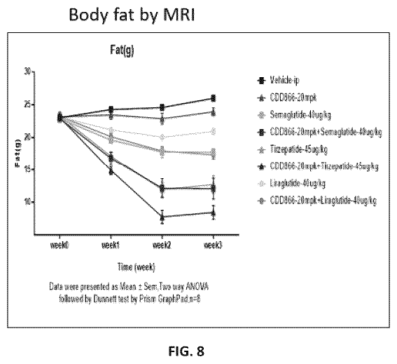

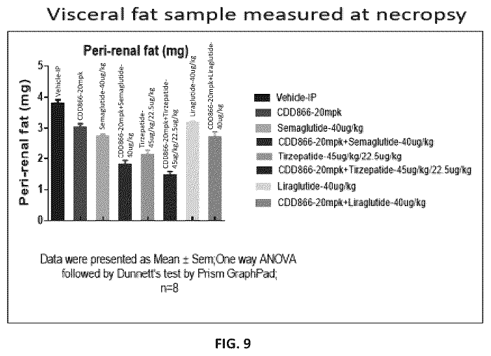

The combination of the ActRII receptor antibody CDD860 with each of the 3 different GLP-1 agonists—semaglutide, liraglutide, and tirzepatide—all yielded greater body weight decreases than either of CDD866 or any of the GLP-1 agonists alone (FIG. 7). The combinations with each GLP-1 agonist yielded at least an additive decrease in body fat, as measured by MRI (FIG. 8), or by weighing peri-renal fat (FIG. 9), measured at necroscopy. Table 4 below, shows that the combination of the ActRII receptor antagonist CDD866 and the GLP-1 agonist tirzepatide resulted in a significantly synergistic reduction in fat mass loss, while still increasing lean mass relative to the control.

TABLE 4
Effects of the combinations of CDD866 with
semaglutide or tirzepatide by day 22

| | Body weight | | Lean |
|---|---|---|---|
| Day 22 | (g) | Fat (g) | mass(g) |
| Vehicle | 56.8 ± 0.7 | 25.6 ± 0.3 | 27.9 ± 0.7 |
| CDD866 (20 mg/kg) | 57.8 ± 1.0 | 23.9 ± 0.6 | 31.9 ± 0.5 |
| Semaglutide (40 μg/kg) | 45.35 ± 1.1 | 17.8 ± 0.5 | 25.8 ± 1.0 |
| CDD866 + Semaglutide | 43.89 ± 1.4 | 12.1 ± 1.6 | 29.7 ± 0.6 |
| Tirzepatide (45/22.5 μg/kg) | 40.2 ± 1.3 | 12.7 ± 1.4 | 25.4 ± 0.4 |
| CDD866 + Tirzepatide | 38.9 ± 1.3 | 8.5 ± 1.0 | 28.8 ± 0.5 |

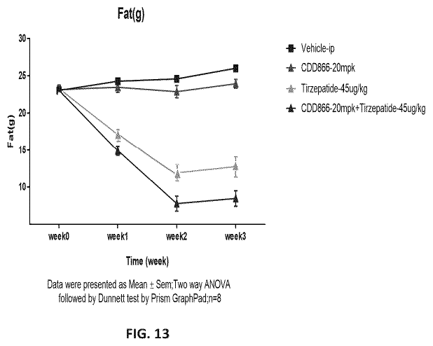

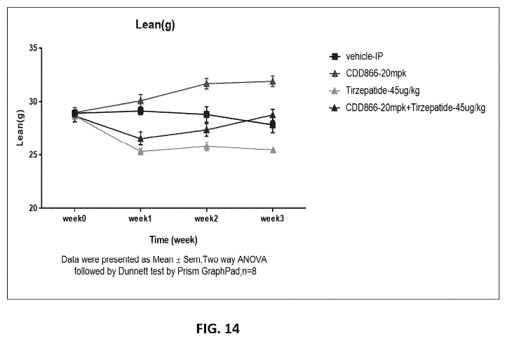

FIGS. 13 and 14 show the significant effects of the combination of CDD800 and tirzepatide on fat mass loss and lean mass maintenance and/or increase.

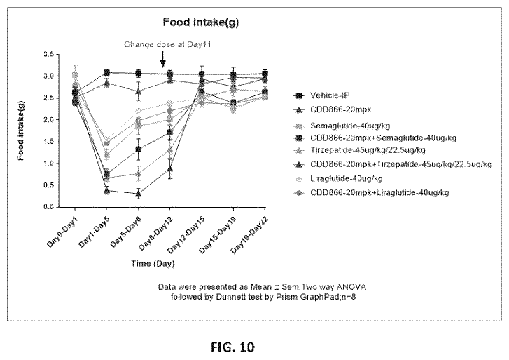

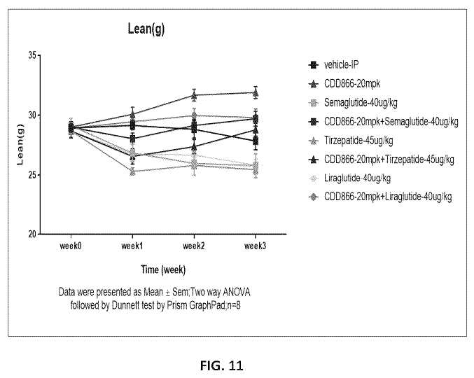

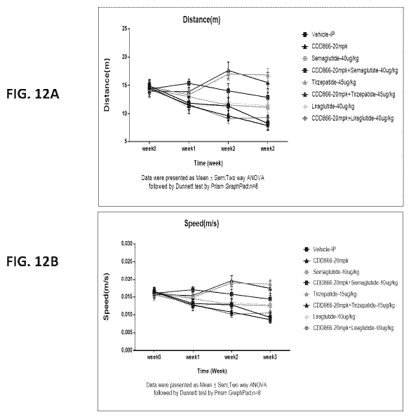

The combination of CDD866 and each of the GLP-1 agonists also significantly reduced food intake, which is associated with fat loss (FIG. 10). Although lean mass increase was expected to be minimal when caloric intake was limited CDD866 in combination with each of the GLP-1 agonists showed a reduction in lean mass loss relative to the GLP-1 agonist alone (FIG. 11). In fact, mice treated with CDD866 in combination with each of the GLP-1 agonists also demonstrated longer open field distances and faster travel speeds (FIGS. 12A and 12B). Overall, fat mass loss and decreased lean mass loss correlated with more activity in the mice.

1. A method of treating a metabolic disorder in a subject, comprising administering to the subject in need thereof an ActRII receptor antibody and a glucagon-like peptide-1 receptor (GLP-1) agonist.

2. The method of claim 1, wherein the metabolic disorder is selected from the group consisting of: obesity, diabetes, metabolic syndrome, anti-psychotic drug-associated obesity, glucocorticoid-induced obesity, hypothalamic obesity associated with craniopharyngioma, Prader-Willi syndrome, and a monogenetic disorder associated with obesity.

3. The method of claim 2, wherein the monogenetic disorder associated with obesity, is one of Bardet-Biedl syndrome, or obesity resulting from mutations in one or more of the genes comprising: ADCY3, ALMS 1, ARL6, BBS 1, BBS2, BBS4, BBS5, BBS7, BBS9, BBS 10, BBS 12, BDNF, CCDC28B, CEP290, CREBBP, EP 300, GNAS, IER3IP1, MC3R, MKKS, MKS1, MRAP2, NTRK2, PCSK1, PHF6, POMC, SH2B1, SIM1, TMEM67, TRIM32, TTC8 and VPS13B.

4. The method of claim 2, wherein the diabetes is Type I diabetes or Type II diabetes.

5. The method of claim 2, wherein the treatment is useful for an obesity related co-morbidity, wherein the condition is selected from the group of: glucose intolerance, prediabetes, insulin resistance, high triglycerides, overweight associated physical impairment, osteoporosis, renal disease, obstructive sleep apnea, sexual hormones impairment, endocrine reproductive disorders, osteoarthritis, gastrointestinal cancers, dyslipidemia, hypertension, heart failure, coronary heart disease, stroke, and/or gallstones.

6. The method of claim 1, wherein the subject has a body mass index (BMI) of 30 or greater.

7. The method of claim 1, wherein the subject has a BMI of 27 or greater and has one or more obesity-related co-morbidities.

8. The method of claim 1, wherein the subject is overweight.

9. The method of claim 1, wherein the subject is 18 years of age or older.

10. The method of claim 1, wherein the subject is 45 years of age or older.

11. The method of claim 1, wherein the subject is a child 0-17 years of age, inclusive.

12. The method of claim 1, wherein the treatment reduces body weight in the subject.

13. The method of claim 1, wherein the treatment reduces fat mass in the subject.

14. The method of claim 1, wherein the treatment increases lean mass in the subject.

15. The method of claim 1, wherein the treatment reduces fat mass and increases lean mass in the subject.

16. The method of claim 1, wherein the treatment reduces fat mass and maintains lean mass in the subject.

17. The method of claim 1, wherein the treatment reduces central adiposity in the subject.

18. The method of claim 1, wherein the treatment improves glycemic control in the subject.

19. The method of claim 1, wherein the treatment improves the safety, efficacy, and/or tolerability of the ActRII receptor antibody and/or the glucagon-like peptide-1 receptor (GLP-1) agonist.

20. The method of claim 1, wherein the efficacy of the treatment is measured by at least one of the following: body weight; bioelectrical impedance analysis (BIA); dual X-ray absorptiometry (DXA); waist circumference; decreased BMI; waist to hip ratio; weight to height ratio; blood lipids profile; leptin, adiponectin, and adipsin levels; urine biomarkers; hemoglobin A1c (HgbA1c) levels; hand dynamometry demonstrating muscle strength; glucose levels; insulin levels; short physical performance battery (SPPB); Impact of Weight on Quality of Life (IWQoL-Lite for CT) assessment; Short Form (36) Health Survey (SF-36) assessment; homeostasis model assessment 2 (HOMA2); and physical activity monitoring via actigraphy.

21. The method of claim 1, wherein the antibody comprises the complementarity determining region (CDR) amino acid sequences of SEQ ID NOS: 1-6.

22. The method of claim 1, wherein the antibody comprises the variable heavy chain amino acid sequence of SEQ ID NO: 7, or a sequence with sequence identity of at least 80% thereto; and/or comprises the variable light chain amino acid sequence of SEQ ID NO: 8, or a sequence with sequence identity of at least 80% thereto.

23. The method of claim 1, wherein the antibody comprises the heavy chain amino acid sequence of SEQ ID NO: 9, or a sequence with sequence identity of at least 80% thereto; and/or comprises the light chain amino acid sequence of SEQ ID NO: 10, or a sequence with sequence identity of at least 80% thereto.

24. The method of claim 1, wherein the antibody is specific for ActRIIA and ActRIIB.

25. The method of claim 1, wherein the GLP-1 agonist is an antibody, small molecule, peptide, or aptamer.

26. The method of claim 25, wherein the GLP-1 agonist is selected from the group consisting of exenatide, exenatide extended-release, dulaglutide, liraglutide, lixisenatide, semaglutide, tirzepatide, cotadutide, noiiglutide, oxyntomodulin, retatrutide, albiglutide, beinaglutide and PEG-loxenatide, pemvidutide, and danuglipron.

27. The method of claim 25, wherein the GLP-1 agonist is a dual GLP-1 agonist and GIP agonist.

28. The method of claim 25, wherein the GLP-1 agonist is a dual GLP-1 agonist and GCG agonist.

29. The method of claim 25, wherein the GLP-1 agonist is a triagonist of GIP/GLP-1/glucagon receptors.

30. The method of claim 1, wherein the ActRII receptor antibody is administered in a dose of about 3 mg/kg to about 50 mg/kg.

31. (canceled)

32. The method of claim 30, wherein the ActRII receptor antibody is administered in a dose of about 10 mg/kg.

33. (canceled)

34. The method of claim 1, wherein the ActRII receptor antibody is administered in a dose of about 200 mg to about 400 mg.

35. The method of claim 34, wherein the ActRII receptor antibody is administered once weekly.

36. The method of claim 1, wherein the ActRII receptor antibody administration includes the administration of at least one loading dose of an ActRII receptor antibody at day 0 or week 0, prior to the administration of the ActRII receptor antibody and the GLP-1 agonist.

37. The method of claim 36, wherein the loading dose of the ActRII receptor antibody is administered in a dose of about 10 mg/kg to about 30 mg/kg.

38. The method of claim 1, wherein the ActRII receptor antibody is administered at least once a day, at least once a week, at least twice a week, at least thrice a week, at least once every 2 weeks, at least once every 4 weeks, at least once every 6 weeks, or at least once every 12 weeks.

39. The method of claim 1, wherein the ActRII receptor antibody is administered at week 0, at about week 4, and at least once every 12 weeks thereafter.

40. The method of claim 1, wherein the GLP-1 agonist is administered in a dose of about 0.005 mg to about 3.0 mg weekly.

41. The method of claim 40, wherein the GLP-1 agonist is administered in a dose of about 0.5 mg.

42. The method of claim 40, wherein the GLP-1 agonist is administered in a dose of about 1.0 mg.

43. The method of claim 40 wherein the GLP-1 agonist dose is administered in a dose of about 1.7 mg.

44. The method of claim 1, wherein the GLP-1 agonist is administered in a dose of about 5.0 mg, about 10 mg, or about 15 mg.

45. The method of claim 1, wherein the ActRII receptor antibody and/or GLP-1 agonist are administered intravenously.

46. The method of claim 1, wherein the ActRII receptor antibody and/or GLP-1 agonist are administered subcutaneously.

47. The method of claim 1, wherein the ActRII receptor antibody is administered prior to the GLP-1 agonist.

48. The method of claim 47, wherein the ActRII receptor antibody is administered at least 12 weeks prior, at least 10 weeks prior, at least 8 weeks prior, at least 6 weeks prior, at least 4 weeks prior, at least 2 weeks prior, at least 1 week prior, at least 1 day prior, or at least 1 hour prior to the administration of the GLP-1 agonist.

49. The method of claim 1, wherein the GLP-1 agonist is administered prior to the ActRII receptor antibody.

50. The method of claim 49, wherein the GLP-1 agonist is administered at least 2 weeks prior, at least 1 week prior, at least 5 days prior, at least 4 days prior, at least 2 days prior, at least 1 day prior, at least 6 hours prior, or at least 1 hour prior to the ActRII receptor antibody.

51. The method of claim 1, wherein the ActRII receptor antibody and the GLP-1 agonist are co-administered.

52. The method of claim 1, wherein the subject is human.

53. A combination comprising an ActRII receptor antibody and a GLP-1 agonist, wherein the ActRII receptor antibody comprises:
(i) the complementarity determining region (CDR) amino acid sequence of SEO ID NOS: 1-6; and/or
(ii) the variable heavy chain amino acid sequence of SEO ID NO: 7, or a sequence with sequence identity of at least 90% thereto, and the variable light chain amino acid sequence of SEO ID NO: 8, or a sequence with sequence identity of at least 90% thereto.

54-55. (canceled)

56. The combination of claim 53, wherein the ActRII receptor antibody comprises the heavy chain amino acid sequence of SEQ ID NO: 9, or a sequence with sequence identity of at least 90% thereto; and comprises the light chain amino acid sequence of SEQ ID NO: 10, or a sequence with sequence identity of at least 90% thereto.

57. The combination of claim 53, wherein the ActRII receptor antibody comprises a human IgG1 Fc domain with a modification selected from the group consisting of 2591, 252Y, 307Q, 308F, 428L, 434H, 434F, 434Y, 434A, 434M, and 434S, relative to a human IgG1 Fc domain according to the EU numbering scheme.

58. The combination of claim 53, wherein the ActRII receptor antibody comprises a human IgG1 Fc domain with a modification selected from M428L and/or N434S relative to a human IgG1 Fc domain according to the EU numbering scheme.

59. The combination of claim 53, wherein the antibody is specific for ActRIIA and ActRIIB.

60. The combination of claim 53, wherein the GLP-1 agonist is selected from the group consisting of exenatide, exenatide extended-release, dulaglutide, liraglutide, lixisenatide, semaglutide, tirzepatide, cotadutide, noiiglutide, oxyntomodulin, retatrutide, albiglutide, beinaglutide and PEG-loxenatide, pemvidutide, and danuglipron.

61. The combination of claim 53, wherein the GLP-1 agonist is a dual GLP-1 agonist and GIP agonist, a dual GLP-1 agonist and GCG agonist, or a triagonist of GIP/GLP-1/glucagon receptors.

62. A pharmaceutical composition comprising the combination of claim 53 and a pharmaceutically acceptable excipient.

63. The combination of claim 61, wherein the dual GLP-1 agonist and GIP agonist is tirzepatide.

64. The combination of claim 60, wherein the GLP-1 agonist is semaglutide.

65. A method of treating a metabolic disorder in a subject, comprising administering to the subject in need thereof an ActRII receptor antibody and a GLP-1 agonist, wherein
(i) the ActRII receptor antibody comprises:
(a) the complementarity determining region (CDR) amino acid sequence of SEQ ID NOS: 1-6; and/or
(b) the variable heavy chain amino acid sequence of SEQ ID NO: 7, or a sequence with sequence identity of at least 90% thereto; and the variable light chain amino acid sequence of SEQ ID NO: 8, or a sequence with sequence identity of at least 90% thereto; and wherein
(ii) the GLP-1 agonist is a dual GLP-1 agonist and GIP agonist.

66. The method of claim 65, wherein the dual GLP-1 agonist and GIP agonist is tirzepatide.

67. A method of treating a metabolic disorder in a subject, comprising administering to the subject in need thereof an ActRII receptor antibody and a GLP-1 agonist, wherein:
(i) the ActRII receptor antibody comprises:
(a) the complementarity determining region (CDR) amino acid sequence of SEQ ID NOS: 1-6; and/or
(b) the variable heavy chain amino acid sequence of SEQ ID NO: 7, or a sequence with sequence identity of at least 90% thereto; and the variable light chain amino acid sequence of SEQ ID NO: 8, or a sequence with sequence identity of at least 90% thereto; and wherein
(ii) the GLP-1 agonist is semaglutide.

68. The method of claim 40, wherein the GLP-1 agonist is administered in a dose of about 2.4 mg.
